(19) United States
(12) Patent Application Publication
(10) Pub. No.: US 2025/0360068 A1
(43) Pub. Date: Nov. 27, 2025

(54) TOPICAL DIM COMPOSITIONS AND COSMETIC USES THEREOF

(71) Applicant: IIAA LIMITED, Hertfordshire (GB)

(72) Inventors: David Alpert, Hertfordshire (GB); Gabriell Prinsloo, San Diego, CA (US)

(21) Appl. No.: 19/218,201

(22) Filed: May 23, 2025

Related U.S. Application Data
(60) Provisional application No. 63/651,889, filed on May 24, 2024.

(51) Int. Cl. A61K 8/49; A61Q 19/00

(52) U.S. Cl. CPC A61K 8/492; A61Q 19/00

(57) ABSTRACT

Described herein are topical compositions for cosmetic use in a subject in need thereof, wherein the composition comprises emulsified substituted or unsubstituted diindolylmethane and a pharmaceutically acceptable carrier, and wherein the composition does not cause irritation or allergic reaction after topical application. In addition, methods are provided for improving the dermal bioavailability of substituted or unsubstituted diindolylmethane through topical administration, relative to other route of administration such as oral administration.

### CROSS REFERENCE TO RELATED APPLICATIONS

This application claims the benefit of U.S. provisional application Ser. No. 63/651,889, filed on May 24, 2024, which is hereby incorpoeated by reference in its entirety.

### BACKGROUND OF THE DISCLOSURE

The FDA defines a cosmetic as a product (excluding pure soap) intended to be applied to the human body for cleansing, beautifying, promoting attractiveness, or altering the appearance. A separate category exists for medications, which are intended to diagnose, cure, mitigate, treat, or prevent disease, or to affect the structure or function of the body.

Though there are a large number of different cosmetics used for a variety of different purposes, all cosmetics are typically intended to be applied to the face, to the body, and/or to the hair. These products may be intended for use as skincare, personal care or to alter the appearance. Sometimes, costmetics are distinguishable between “decorative” cosmetics intended to alter the appearance and “care” cosmetics designed for skincare and personal care. Skin care or skincare is a range of practices that support skin integrity, enhance skin appearance, relieve skin stress, and/or slow down skin aging.

### SUMMARY OF THE DISCLOSURE

One aspect of the present disclosure provides a topical composition for cosmetic use in a subject in need thereof, wherein the composition comprises substituted or unsubstituted diindolylmethane and a pharmaceutically acceptable carrier, and wherein the composition does not cause irritation or allergic reaction after topical application. In some embodiments, the substituted or unsubstituted diindolylmethane is at least partially emulsified in the composition. In some embodiments, the substituted or unsubstituted diindolylmethane is fully emulsified in the composition. In some embodiments, the substituted or unsubstituted diindolylmethane is at least partially dissolved in the composition. In some embodiments, the substituted or unsubstituted diindolylmethane is fully dissolved in the composition. In some embodiments, the substituted or unsubstituted diindolylmethane is at least partially suspended in the composition. In some embodiments, the substituted or unsubstituted diindolylmethane is fully suspended in the composition.

One aspect of the present disclosure provides a topical composition for cosmetic use in a subject in need thereof, wherein the composition comprises emulsified substituted or unsubstituted diindolylmethane and a pharmaceutically acceptable carrier, and wherein the composition does not cause irritation or allergic reaction after topical application.

One aspect of the present disclosure provides a topical composition for cosmetic use in a subject in need thereof, wherein the composition comprises from about 0.1 wt % to about 10 wt % of substituted or unsubstituted diindolylmethane and a pharmaceutically acceptable carrier, and wherein the composition does not cause irritation or allergic reaction after topical application.

One aspect of the present disclosure provides a topical composition for cosmetic use in a subject in need thereof, wherein the composition comprises from about 0.1 wt % to about 10 wt % of emulsified substituted or unsubstituted diindolylmethane and a pharmaceutically acceptable carrier, and wherein the composition does not cause irritation or allergic reaction after topical application.

In some embodiments, the cosmetic use comprises supporting skin integrity.

In some embodiments, the cosmetic use comprises enhancing skin appearance.

In some embodiments, the cosmetic use comprises relieving skin stress.

In some embodiments, the cosmetic use comprises slowing down skin aging.

In some embodiments, the cosmetic use comprises reducing the number and/or severity of wrinkles.

In some embodiments, the cosmetic use comprises reducing the number and/or severity of fine lines.

In some embodiments, the cosmetic use comprises reducing skin laxity.

In some embodiments, the cosmetic use comprises reducing the number, area, and/or darkness of age spots.

In some embodiments, the cosmetic use comprises reducing the number, area, and/or darkness of stretch marks.

In some embodiments, the cosmetic use comprises reducing the number, area, and/or darkness of spider veins.

In some embodiments, the cosmetic use comprises reducing skin dryness.

In some embodiments, the cosmetic use comprises reducing skin oil buildup.

In some embodiments, the cosmetic use comprises repairing sun damage.

In some embodiments, the cosmetic use comprises alleviating peeling skin.

In some embodiments, the cosmetic use comprises alleviating scaling skin.

In some embodiments, the cosmetic use comprises improving skin tone.

In some embodiments, the cosmetic use comprises reducing skin dullness.

In some embodiments, the cosmetic use comprises reducing appearance of mole.

In some embodiments, the cosmetic use comprises reducing appearance of scar.

In some embodiments, the cosmetic use comprises reducing appearance of birthmark.

In some embodiments, the cosmetic use comprises reducing skin blushing or skin flushing.

In some embodiments, the cosmetic use comprises reducing skin flushing.

In some embodiments, the cosmetic use comprises reducing under-eye dark circle.

In some embodiments, the cosmetic use comprises reducing under-eye bag.

In some embodiments, the cosmetic use comprises skin lightening.

In some embodiments, the cosmetic use comprises skin brightening.

In some embodiments, the cosmetic use is selected from reducing the number and/or severity of wrinkles, reducing the number and/or severity of fine lines, reducing skin laxity, reducing the number, area, and/or darkness of age spots, reducing the number, area, and/or darkness of stretch marks, reducing the number, area, and/or darkness of spider veins, reducing skin dryness, reducing skin oil buildup, repairing sun damage, alleviating peeling skin, alleviating scaling skin, improving skin tone, reducing skin dullness, reducing appearance of mole, reducing appearance of scar, reducing appearance of birthmark, reducing skin blushing or skin flushing, reducing under-eye dark circle, reducing under-eye bag, skin lightening, skin brightening, or combinations thereof.

In some embodiments, the cosmetic use is non-medical use.

In some embodiments, the cosmetic use does not comprise or involve treatment of a skin diseases or skin disorder.

In some embodiments, the topical composition further comprises a retinoid compound. In some embodiments, the retinoid compound is a substituted or unsubstituted retinoic acid. In some embodiments, the retinod compound is vitamin A. In some embodiments, the retinoid compound is retinyl palmitate.

In some embodiments, the topical composition is essentially free of any retinoid compound, such as retinoid acid, vitamin A or retinyl palmitate. In some embodiments, the topical composition is free of any retinoid compound, such as retinoid acid, vitamin A or retinyl palmitate. In some embodiments, the topical composition does not any retinoid compound, such as retinoid acid, vitamin A or retinyl palmitate. In some embodiment, the topical composition does not comprise any additional therapeutically active agent other than the substituted or unsubstituted diindolylmethane.

In some embodiments, the composition comprises from about 0.1 wt % to about 9 wt % of the substituted or unsubstituted diindolylmethane. In some embodiments, the composition comprises from about 0.1 wt % to about 8 wt % of the substituted or unsubstituted diindolylmethane. In some embodiments, the composition comprises from about 0.1 wt % to about 7 wt % of the substituted or unsubstituted diindolylmethane. In some embodiments, the composition comprises from about 0.1 wt % to about 6 wt % of the substituted or unsubstituted diindolylmethane. In some embodiments, the composition comprises from about 0.1 wt % to about 5 wt % of the substituted or unsubstituted diindolylmethane. In some embodiments, the composition comprises from about 0.1 wt % to about 4 wt % of the substituted or unsubstituted diindolylmethane. In some embodiments, the composition comprises from about 0.1 wt % to about 3 wt % of the substituted or unsubstituted diindolylmethane. In some embodiments, the composition comprises from about 0.1 wt % to about 2.5 wt % of the substituted or unsubstituted diindolylmethane.

The topical composition of any one of claims 1-108, wherein the composition comprises from about 0.3 wt % to about 0.8 wt % of substituted or unsubstituted diindolylmethane. In some embodiments, the composition comprises about 0.5 wt % of substituted or unsubstituted diindolylmethane.

In some embodiments, the composition comprises from about 0.8 wt % to about 1.2 wt % of substituted or unsubstituted diindolylmethane. In some embodiments, the composition comprises from about 1.0 wt % of substituted or unsubstituted diindolylmethane.

In some embodiments, the composition comprises from about 1.2 wt % to about 2.0 wt % of substituted or unsubstituted diindolylmethane. In some embodiments, the composition comprises about 1.5 wt % of substituted or unsubstituted diindolylmethane.

In some embodiments, the composition comprises from about 2.0 wt % to about 3.0 wt % of substituted or unsubstituted diindolylmethane. In some embodiments, the composition comprises about 2.5 wt % of substituted or unsubstituted diindolylmethane.

In some embodiments, the composition comprises from about 0.3 wt % to about 9 wt % of the substituted or unsubstituted diindolylmethane. In some embodiments, the composition comprises from about 0.3 wt % to about 8 wt % of the substituted or unsubstituted diindolylmethane. In some embodiments, the composition comprises from about 0.3 wt % to about 7 wt % of the substituted or unsubstituted diindolylmethane. In some embodiments, the composition comprises from about 0.3 wt % to about 6 wt % of the substituted or unsubstituted diindolylmethane. In some embodiments, the composition comprises from about 0.3 wt % to about 5 wt % of the substituted or unsubstituted diindolylmethane. In some embodiments, the composition comprises from about 0.3 wt % to about 4 wt % of the substituted or unsubstituted diindolylmethane. In some embodiments, the composition comprises from about 0.3 wt % to about 3 wt % of the substituted or unsubstituted diindolylmethane. In some embodiments, the composition comprises from about 0.3 wt % to about 2.5 wt % of the substituted or unsubstituted diindolylmethane.

In some embodiments, the composition comprises from about 0.5 wt % to about 9 wt % of the substituted or unsubstituted diindolylmethane. In some embodiments, the composition comprises from about 0.5 wt % to about 8 wt % of the substituted or unsubstituted diindolylmethane. In some embodiments, the composition comprises from about 0.5 wt % to about 7 wt % of the substituted or unsubstituted diindolylmethane. In some embodiments, the composition comprises from about 0.5 wt % to about 6 wt % of the substituted or unsubstituted diindolylmethane. In some embodiments, the composition comprises from about 0.5 wt % to about 5 wt % of the substituted or unsubstituted diindolylmethane. In some embodiments, the composition comprises from about 0.5 wt % to about 4 wt % of the substituted or unsubstituted diindolylmethane. In some embodiments, the composition comprises from about 0.5 wt % to about 3 wt % of the substituted or unsubstituted diindolylmethane. In some embodiments, the composition comprises from about 0.5 wt % to about 2.5 wt % of the substituted or unsubstituted diindolylmethane.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates the structure of skin, particularly showing the epidermis, dermis and hypodermis layers; and

FIG. 2 schemattically illustrate the microdialysis and tape striping technique.

FIG. 3 illustrates the effect of topical DIM on reduction of redness/erythema.

FIG. 4 illustrates a comparison of reduction of pigmentation/skin tone between topical DIM and no DIM groups.

FIG. 5 illustrates additional examples of reduction of pigmentation/skin tone by topical DIM.

FIG. 6 illustrates examples of improvement of skin texture and smoothness by topical DIM.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed. It is intended that the following claims define the scope of the present disclosure and that methods and structures within the scope of these claims and their equivalents be covered thereby.

Unless defined otherwise, all technical and scientific terms used herein have the same meaning as commonly understood by one of skill in the art to which the present disclosure described herein belong. All publications, patents, and patent applications mentioned in this specification are hereby incorporated by reference to the same extent as if each individual publication, patent, or patent application was specifically and individually indicated to be incorporated by reference.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Certain Definitions

Unless defined otherwise, all technical and scientific terms used herein have the same meaning as is commonly understood by one of skill in the art to which the claimed subject matter belongs. In the event that there is a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

As used herein, the term “about” is used synonymously with the term “approximately.” Illustratively, the use of the term “about” with regard to a certain therapeutically effective pharmaceutical dose indicates that values slightly outside the cited values, e.g., plus or minus 0.1% to 10%, are also effective and safe.

The term “subject” or “individual” are used interchangeably. As used herein, they refer to individuals that could benefit from a costmetic product. None of the terms require that the individual be under the care and/or supervision of a medical professional. In preferred embodiments, the individual is a human.

The terms “administer,” “administering”, “administration,” and the like, as used herein, refer to the methods that may be used to enable delivery of compounds or compositions to the desired site of biological action. These methods include, but are not limited to topical routes, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein.

The term “acceptable” as used herein, with respect to a formulation, composition or ingredient, means having no persistent detrimental effect on the general health of the individual being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “DIM” as used herein, refers to a substituted or unsubstituted diindolylmethane compound.

The term “BR-DIM” or “BioResponse DIM” as used herein, refers to an unsubstituted DIM, provided by BioResponse LLC.

The term “maximum concentration” or Cmax as used herein, refers to the Cmax refers to the maximum (or peak) serum concentration that the substituted or unsubstituted diindolylmethane achieves in the plasma after it has been administrated and prior to the administration of a second dose.

The term “time to maximum concentration” or Tmax as used herein, refers to the time at which the Cmax is observed.

The term “area under the curve” or AUC or AUC_{0-inf} as used herein, refers to the area under the curve, also known as the definite integral, in a plot of concentration of cosmetic agent (e.g. DIM) in blood plasma against time.

The term “unit dose” as used herein, refers to an amount of substituted or unsubstituted diindolylmethane contained in one discreet pharmaceutical dosage form. Examples of pharmaceutical dosage form that contains a unit dose include but are not limited to a tablet, a capsule, a buccal tablet, a sub-lingual tablet, an orally-disintegrating tablet, an effervescent tablet, a lollipop, a lozenge, a troche, a liquid solution or suspension, powder or liquid or solid crystals packed within a single tablet or capsule, a cream, a gel, an ointment, a lotion.

DIM

Diindolylmethane (DIM) is a natural compound formed during the autolytic breakdown of glucobrassicin present in food plants of the Brassica genus, including broccoli, cabbage, Brussels sprouts, cauliflower and kale. The autolytic breakdown of glucobrassicin requires the catalytic reaction of the enzyme myrosinase, which is endogenous to these plants and released upon rupture of the cell wall. The compound is normally manufactured by chemical synthesis but in some embodiments is also prepared by natural means from the extracts of Brassica vegetables, as listed above, particularly from sprouting broccoli or from broccoli seeds.

Thus, the substituted or unsubstituted DIM in some embodiments is synthetic, or in some embodiments is a natural product obtained from a Brassica plant, as discussed above.

In some embodiments, the composition described herein comprises a diindolylmethane of Formula 1:

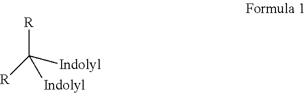

wherein the R groups are independently selected from hydrogen atoms and C1-C6 hydrocarbon substituents; and wherein the indolyl groups are independently selected from indole-3-yl and indole-2-yl groups; and wherein the indolyl groups are unsubstituted, or are substituted with one or more C1-C6 hydrocarbon substituents.

In some embodiments, the composition described herein comprises an unsubstituted DIM. In some embodiments, the composition described herein comprises an unsubstituted 3,3′diindolylmethane.

In some embodiments, the composition described herein comprises encapsulated DIM to further improve the bioavailability of DIM. In some embodiments, the encapsulated DIM is BR-DIM.

### Skin Aging

Aging changes in the skin are a group of common conditions and developments that occur as people grow older. Skin changes are among the most visible signs of aging. Evidence of increasing age includes wrinkles and sagging skin. Whitening or graying of the hair is another obvious sign of aging.

As shown in FIG. 1, skin has many layers and can generally be divided into three main parts: the outer part (epidermis) contains skin cells, pigment, and proteins; the middle part (dermis) contains skin cells, blood vessels, nerves, hair follicles, and oil glands; and the inner layer under the dermis (the subcutaneous layer or hypodermis) contains sweat glands, some hair follicles, blood vessels, and fat. Each layer also contains connective tissue with collagen fibers to give support and elastin fibers to provide flexibility and strength.

Skin changes are related to environmental factors, genetic makeup, nutrition, and other factors. The greatest single factor, though, is sun exposure. Natural pigments seem to provide some protection against sun-induced skin damage. Fair-skinned people sometimes show more aging skin changes than people with darker, more heavily pigmented skin.

With aging, the outer skin layer (epidermis) thins, even though the number of cell layers remains unchanged. The number of pigment-containing cells (melanocytes) decreases. The remaining melanocytes increase in size. Aging skin looks thinner, paler, and clear (translucent). Pigmented spots including age spots or “liver spots” may appear in sun-exposed areas. The medical term for these areas is lentigos.

Changes in the connective tissue reduce the skin's strength and elasticity. This is known as elastosis. It is more noticeable in sun-exposed areas (solar elastosis). Elastosis produces the leathery, weather-beaten appearance common to farmers, sailors, and others who spend a large amount of time outdoors. The blood vessels of the dermis become more fragile. This leads to bruising, bleeding under the skin (often called senile purpura), and cherry angiomas. Sebaceous glands produce less oil as you age. Men experience a minimal decrease, most often after the age of 80. Women gradually produce less oil beginning after menopause. This can make it harder to keep the skin moist, resulting in dryness and itchiness.

Growths such as skin tags, warts, brown rough patches (seborrheic keratoses), and other blemishes are more common in older people.

Without wishing to be bound by any particular theory, it is contemplated that the DIM-based topical composition discloses herein, in some embodiments, slows down skin aging better than other costmetic products/agents, or without the adverse effects (e.g. skin irritation and allergic reaction) of other cosmtic products/agents, or both.

### Skin Scarring

A scar (or scar tissue) is an area of fibrous tissue that replaces normal skin after an injury. Scars result from the biological process of wound repair in the skin, as well as in other organs, and tissues of the body. Thus, scarring is a natural part of the healing process. With the exception of very minor lesions, every wound (e.g., after accident, disease, or surgery) results in some degree of scarring. An exception to this are animals with complete regeneration, which regrow tissue without scar formation.

Scar tissue is composed of the same protein (collagen) as the tissue that it replaces, but the fiber composition of the protein is different; instead of a random basketweave formation of the collagen fibers found in normal tissue, in fibrosis the collagen cross-links and forms a pronounced alignment in a single direction. [1] This collagen scar tissue alignment is usually of inferior functional quality to the normal collagen randomised alignment. For example, scars in the skin are less resistant to ultraviolet radiation, and sweat glands and hair follicles do not grow back within scar tissues. A myocardial infarction, commonly known as a heart attack, causes scar formation in the heart muscle, which leads to loss of muscular power and possibly heart failure. However, there are some tissues (e.g. bone) that can heal without any structural or functional deterioration.

Scarring is generally composed of the same collagen as the tissue it has replaced, but the composition of the scar tissue, compared to the normal tissue, is different. Scar tissue also lacks elasticity unlike normal tissue which distributes fiber elasticity. Scars differ in the amounts of collagen overexpressed. Labels have been applied to the differences in overexpression. Two of the most common types are hypertrophic and keloid scarring, [4] both of which experience excessive stiff collagen bundled growth overextending the tissue, blocking off regeneration of tissues. Another form is atrophic scarring (sunken scarring), which also has an overexpression of collagen blocking regeneration. This scar type is sunken, because the collagen bundles do not overextend the tissue. Stretch marks (striae) are regarded as scars by some. High melanin levels and either African or Asian ancestry may make adverse scarring more noticeable.

### Hypertrophic

Hypertrophic scars occur when the body overproduces collagen, which causes the scar to be raised above the surrounding skin. Hypertrophic scars take the form of a red raised lump on the skin for lighter pigmented skin and the form of dark brown for darker pigmented skin. They usually occur within 4 to 8 weeks following wound infection or wound closure with excess tension and/or other traumatic skin injuries.

### Keloid

Keloid scars are a more serious form of excessive scarring, because they can grow indefinitely into large, tumorous (although benign) neoplasms.

Hypertrophic scars are often distinguished from keloid scars by their lack of growth outside the original wound area, but this commonly taught distinction can lead to confusion.

Keloid scars can occur on anyone, but they are most common in dark-skinned people. They can be caused by surgery, cuts, accident, acne or, sometimes, body piercings. In some people, keloid scars form spontaneously. Although they can be a cosmetic problem, keloid scars are only inert masses of collagen and therefore completely harmless and not cancerous. However, they can be itchy or painful in some individuals. They tend to be most common on the shoulders and chest. Hypertrophic scars and keloids tend to be more common in wounds closed by secondary intention. Surgical removal of keloid is risky and may exacerbate the condition and worsening of the keloid.

### Atrophic

An atrophic scar takes the form of a sunken recess in the skin, which has a pitted appearance. These are caused when underlying structures supporting the skin, such as fat or muscle, are lost. This type of scarring is often associated with acne, chickenpox, other diseases (especially Staphylococcus infection), surgery, certain insect and spider bites, or accidents. It can also be caused by a genetic connective tissue disorder, such as Ehlers-Danlos syndrome.

### Stretch Marks

Stretch marks (technically called striae) are also a form of scarring. These are caused when the skin is stretched rapidly (for instance during pregnancy, significant weight gain, or adolescent growth spurts), or when skin is put under tension during the healing process (usually near joints). This type of scar usually improves in appearance after a few years. Elevated corticosteroid levels are implicated in striae development.

Without wishing to be bound by any particular theory, it is contemplated that the DIM-based topical composition discloses herein, in some embodiments, provides skin tightening benefits better than other cosmtic products/agents, or without the adverse effects (e.g. skin irritation and allergic reaction) of other cosmtic products/agents, or both.

### Skin Laxity/Tightening

Skin laxity is a common concern that many individuals experience as they age, lose weight and/or have certain diseases or disorders. It is characterized by a loss of firmness, elasticity, and resilience in the skin, leading to a less youthful appearance.

Skin laxity can affect various areas of the body, including the face, neck, arms, abdomen, and thighs. Skin laxity contributes to skin sagging as the skin loses its elasticity and becomes less able to maintain its natural position against the effects of gravity.

### Causes of Skin Laxity

Natural Aging Process: As we age, the production of collagen and elastin proteins, which are responsible for maintaining the skin's firmness and elasticity, decreases. This leads to a loss of skin support and resilience, resulting in sagging and laxity.

Sun Damage: Prolonged exposure to ultraviolet (UV) radiation from the sun can accelerate the breakdown of collagen and elastin fibers in the skin. This can lead to a reduction in skin elasticity and contribute to the development of skin laxity.

Lifestyle Factors: Unhealthy lifestyle choices, such as smoking, excessive alcohol consumption, poor nutrition, and lack of exercise, can accelerate the aging process and negatively impact the skin's elasticity. These habits can compromise collagen production and contribute to skin laxity.

It's important to note that these factors can interact and vary in intensity for each individual, resulting in differences in the degree of skin laxity experienced. Additionally, environmental factors, stress, and overall health can also play a role in the development of skin laxity.

### Collagen and Elastin

Collagen and elastin are vital proteins that play crucial roles in maintaining the health and appearance of the skin.

Collagen: Collagen is the most abundant protein in the body and acts as the building block for the skin's structure. It provides strength, support, and firmness to the skin. Collagen fibers form a network that gives the skin its structural integrity, preventing it from sagging and maintaining its smoothness. It helps to maintain a youthful appearance by promoting skin elasticity and reducing the appearance of wrinkles and fine lines.

Elastin: Elastin, as the name suggests, is responsible for the skin's elasticity. It allows the skin to stretch and then return to its original shape, helping it maintain its tautness and resilience. Elastin fibers are highly flexible and can recoil after being stretched, enabling the skin to retain its tightness and prevent sagging. Elastin works in conjunction with collagen to provide the skin with the ability to bounce back and maintain its youthful appearance.

### Decline of Collagen and Elastin

The production of collagen and elastin can decline due to aging, weight lose and/or certain diseases or disorders. Several factors contribute to this decline:

Genetic Factors: The genetic makeup of an individual can influence the production and quality of collagen and elastin in the skin. Some people may have genetically determined variations in their collagen and elastin structure, making them more prone to skin laxity and premature aging.

Hormonal Changes: Hormonal fluctuations, particularly during menopause in women, can lead to a decrease in collagen production. Estrogen, a hormone that helps maintain skin elasticity, declines with age, resulting in reduced collagen synthesis and increased skin laxity.

Environmental Influences: External factors such as sun exposure, pollution, and lifestyle habits like smoking and poor nutrition can accelerate the breakdown of collagen and elastin fibers. UV radiation from the sun is particularly damaging, as it can cause the production of harmful enzymes that degrade collagen and elastin.

The decline in collagen and elastin levels contributes to the loss of skin elasticity, firmness, and resilience. The skin becomes more prone to sagging, wrinkles, and fine lines.

Without wishing to be bound by any particular theory, it is contemplated that the topical composition discloses herein, in some embodiments, stimulate collagen and elastin production and/or provide external support to improve skin laxity and maintain a youthful appearance better than other costmetic products/agents, or without the adverse effects (e.g. skin irritation and allergic reaction) of other cosmtic products/agents, or both.

### Skin Whitening/Lightening

Skin whitening, also known as skin lightening and skin bleaching, is the practice of using chemical substances in an attempt to lighten the skin or provide an even skin color by reducing the melanin concentration in the skin. Several chemicals have been shown to be effective in skin whitening, while some have proven to be toxic or have questionable safety profiles. This includes mercury compounds which may cause neurological problems and kidney problems.

Areas of increased pigmentation such as moles may be depigmented to match the surrounding skin. Effective agents for specific areas include corticosteroids, tretinoin, and hydroquinone. These agents, however, are not allowed in cosmetics in Europe due to concerns about side effects. Attempts to whiten large areas of skin may also be carried out by certain cultures. This may be done for reasons of appearance, politics, or economics. Skin whiteners can help achieve lighter skin tones, but many of them contain harmful ingredients like the steroid clobetasol propionate, inorganic mercury (mercuric chloride or amalgamated mercury), glutathione (an antioxidant traditionally used in cancer treatment), and the organic compound hydroquinone. Skin lighteners' main health risks are linked to (i) The overuse of topical clobetasol, which can cause systemic steroid effects from daily usage, especially on broad skin regions; and (ii) concealed mercury content, which can lead to mercury poisoning depending on individual susceptibility. Many skin whiteners contain a toxic form of mercury as the active ingredient. Their use, therefore, may harm a person's health and is illegal in many countries.

Hydroquinone is a commonly used agent in skin whiteners, though the European Union banned it from cosmetics in 2000. It works by decreasing melanin production. Tretinoin, also known as all-trans retinoic acid, may be used to whiten specific areas. It may be used in combination with steroids and hydroquinone.

Alpha hydroxy acid (AHA) is also used as a skin bleacher, but its biochemical mechanism is unclear. Side effects may include sun sensitivity, skin redness, thickening, or itching. Low concentrations may be used in cosmetics. Kojic acid has been found to be an effective lightener in some studies and is also allowed to be used in cosmetics. Side effects, however, include redness and eczema.

Glutathione is the most common agent taken by mouth in an attempt to whiten the skin. It may also be used as a cream. It is an antioxidant normally made by the body. Whether or not it provides skin whitening/lightening benefits is unclear. Due to side effects that may result from intravenous use, the government of the Philippines recommends against such use.

Without wishing to be bound by any particular theory, it is contemplated that the DIM-based topical composition discloses herein, in some embodiments, provides skin whitening/lightening benefits better than other costmetic products/agents, or without the adverse effects (e.g. skin irritation and allergic reaction) of other cosmtic products/agents, or both.

### Cosmetic Uses of Topical DIM Compositions

Provided herein in some embodiments are methods of improving appearance of skin in a subject in need thereof comprising administering to the subject a composition comprising substituted or unsubstituted diindolylmethane, wherein the composition is administered by topical route. In some embodiments, the composition comprising substituted or unsubstituted diindolylmethane further comprises substituted or unsubstituted retinoic acid based component.

In some embodiments, the increase in dermal bioavailability of substituted or unsubstituted diindolylmethane upon delivery by topical route is relative to the dermal bioavailability upon administration by oral route, at the same delivery dose, of the same composition. In some embodiments, the increase in dermal bioavailability of substituted or unsubstituted diindolylmethane upon delivery by topical route is relative to the dermal bioavailability upon administration by oral route, at the same delivery dose, of a comparative composition.

In some embodiments, administration of a composition comprising substituted or unsubstituted diindolylmethane by topical route helps by-pass the gastric juices, acid environment and enzymes present in the gastrointestinal tract, as compared to oral administration. In some embodiments, administration of a composition comprising substituted or unsubstituted diindolylmethane by topical route helps by-pass the saliva and enzymes present in the oral cavity, as compared to sublingual or buccal administration.

In some embodiments, increased dermal bioavailability of the substituted or unsubstituted diindolylmethane by topical administration is attributed to by-passing the gastric juices, acid environment and enzymes present in the gastrointestinal tract. In some embodiments, the administration of substituted or unsubstituted diindolylmethane by topical route helps by-pass the liver which is the target organ for metabolism of the cosmetic agent (e.g. DIM) when administered orally. In some embodiments, increased dermal bioavailability of the substituted or unsubstituted diindolylmethane by topical administration is attributed to by-passing first pass metabolism by the liver. In some embodiments, the highly vascular mucosal lining between the cheek and gum where buccal formulations are placed or under the tongue where sublingual formulations are placed are ideal and convenient locations for the substituted or unsubstituted diindolylmethane to be absorbed.

### Increase in Dermal Bioavailability Of DIM by Topical Administration

In some embodiments, the administration by topical route increases the dermal bioavailability of the substituted or unsubstituted diindolylmethane by about 1-fold to about 1000-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 500-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 100-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 50-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 20-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 10-fold compared to the dermal bioavailability of the substituted or unsubstituted diindolylmethane of a comparative composition administered by oral route at the same delivery dose.

In some embodiments, the administration by topical route increases the dermal bioavailability of the substituted or unsubstituted diindolylmethane by about 1-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 2-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 3-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 4-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 5-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 10-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 20-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 30-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 40-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 50-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 60-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 70-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 80-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 100-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 200-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 300-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 400-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 500-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 600-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 700-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 800-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 900-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1000-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 100-fold compared to the dermal bioavailability of the substituted or unsubstituted diindolylmethane of a comparative composition administered by oral route at the same delivery dose.

### Improved Skin Bioavailability Upon Administration of DIM by Topical Route

Dermal bioavailability includes the following exemplary factors: rate (or time after administration) of achievement of minimum effective cosmetic agent serum concentration (MEC), maximum cosmetic agent serum concentration (Cmax), rate (or time after administration) of achievement of maximum cosmetic agent serum concentration (Tmax), and the area under the cosmetic agent serum concentration-time curve above a line representing minimum effective cosmetic agent serum concentration (AUC). In some embodiments, the methods of improving skin appearance by administration of substituted or unsubstituted diindolylmethane by topical route, as described herein, leads to enhancement in one more of the factors mentioned above.

### Increased Cmax

In some embodiments, following administration of the composition by topical route the Cmax of the substituted or unsubstituted diindolylmethane in skin increases by about 1-fold to about 1000-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 500-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 100-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 50-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 20-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 10-fold compared to the Cmax following administration of a comparative composition by oral route at the same delivery dose.

In some embodiments, following administration of the composition by topical route the Cmax of the substituted or unsubstituted diindolylmethane in skin increases by about 1-fold compared to the Cmax following administration of a comparative composition by oral route at the same delivery dose. In some embodiments, following administration of the composition by topical route the Cmax of the substituted or unsubstituted diindolylmethane in skin increases by about 2-fold compared to the Cmax following administration of a comparative composition by oral route at the same delivery dose. In some embodiments, following administration of the composition by topical route the Cmax of the substituted or unsubstituted diindolylmethane in skin increases by about 3-fold compared to the Cmax following administration of a comparative composition by oral route at the same delivery dose. In some embodiments, following administration of the composition by topical route the Cmax of the substituted or unsubstituted diindolylmethane in skin increases by about 4-fold compared to the Cmax following administration of a comparative composition by oral route at the same delivery dose. In some embodiments, following administration of the composition by topical route the Cmax of the substituted or unsubstituted diindolylmethane in skin increases by about 5-fold compared to the Cmax following administration of a comparative composition by oral route at the same delivery dose. In some embodiments, following administration of the composition by topical route the Cmax of the substituted or unsubstituted diindolylmethane in skin increases by about 6-fold compared to the Cmax following administration of a comparative composition by oral route at the same delivery dose. In some embodiments, following administration of the composition by topical route the Cmax of the substituted or unsubstituted diindolylmethane in skin increases by about 7-fold compared to the Cmax following administration of a comparative composition by oral route at the same delivery dose. In some embodiments, following administration of the composition by topical route the Cmax of the substituted or unsubstituted diindolylmethane in skin increases by about 8-fold compared to the Cmax following administration of a comparative composition by oral route at the same delivery dose. In some embodiments, following administration of the composition by topical route the Cmax of the substituted or unsubstituted diindolylmethane in skin increases by about 9-fold compared to the Cmax following administration of a comparative composition by oral route at the same delivery dose. In some embodiments, following administration of the composition by topical route the Cmax of the substituted or unsubstituted diindolylmethane in skin increases by about 10-fold compared to the Cmax following administration of a comparative composition by oral route at the same delivery dose. In some embodiments, following administration of the composition by topical route the Cmax of the substituted or unsubstituted diindolylmethane in skin increases by about 20-fold compared to the Cmax following administration of a comparative composition by oral route at the same delivery dose. In some embodiments, following administration of the composition by topical route the Cmax of the substituted or unsubstituted diindolylmethane in skin increases by about 30-fold compared to the Cmax following administration of a comparative composition by oral route at the same delivery dose. In some embodiments, following administration of the composition by topical route the Cmax of the substituted or unsubstituted diindolylmethane in skin increases by about 40-fold compared to the Cmax following administration of a comparative composition by oral route at the same delivery dose. In some embodiments, following administration of the composition by topical route the Cmax of the substituted or unsubstituted diindolylmethane in skin increases by about 50-fold compared to the Cmax following administration of a comparative composition by oral route at the same delivery dose. In some embodiments, following administration of the composition by topical route the Cmax of the substituted or unsubstituted diindolylmethane in skin increases by about 60-fold compared to the Cmax following administration of a comparative composition by oral route at the same delivery dose. In some embodiments, following administration of the composition by topical route the Cmax of the substituted or unsubstituted diindolylmethane in skin increases by about 70-fold compared to the Cmax following administration of a comparative composition by oral route at the same delivery dose. In some embodiments, following administration of the composition by topical route the Cmax of the substituted or unsubstituted diindolylmethane in skin increases by about 80-fold compared to the Cmax following administration of a comparative composition by oral route at the same delivery dose. In some embodiments, following administration of the composition by topical route the Cmax of the substituted or unsubstituted diindolylmethane in skin increases by about 90-fold compared to the Cmax following administration of a comparative composition by oral route at the same delivery dose. In some embodiments, following administration of the composition by topical route the Cmax of the substituted or unsubstituted diindolylmethane in skin increases by about 100-fold compared to the Cmax following administration of a comparative composition by oral route at the same delivery dose. In some embodiments, following administration of the composition by topical route the Cmax of the substituted or unsubstituted diindolylmethane in skin increases by about 200-fold compared to the Cmax following administration of a comparative composition by oral route at the same delivery dose. In some embodiments, following administration of the composition by topical route the Cmax of the substituted or unsubstituted diindolylmethane in skin increases by about 300-fold compared to the Cmax following administration of a comparative composition by oral route at the same delivery dose. In some embodiments, following administration of the composition by topical route the Cmax of the substituted or unsubstituted diindolylmethane in skin increases by about 400-fold compared to the Cmax following administration of a comparative composition by oral route at the same delivery dose. In some embodiments, following administration of the composition by topical route the Cmax of the substituted or unsubstituted diindolylmethane in skin increases by about 500-fold compared to the Cmax following administration of a comparative composition by oral route at the same delivery dose. In some embodiments, following administration of the composition by topical route the Cmax of the substituted or unsubstituted diindolylmethane in skin increases by about 600-fold compared to the Cmax following administration of a comparative composition by oral route at the same delivery dose. In some embodiments, following administration of the composition by topical route the Cmax of the substituted or unsubstituted diindolylmethane in skin increases by about 700-fold compared to the Cmax following administration of a comparative composition by oral route at the same delivery dose. In some embodiments, following administration of the composition by topical route the Cmax of the substituted or unsubstituted diindolylmethane in skin increases by about 800-fold compared to the Cmax following administration of a comparative composition by oral route at the same delivery dose. In some embodiments, following administration of the composition by topical route the Cmax of the substituted or unsubstituted diindolylmethane in skin increases by about 800-fold compared to the Cmax following administration of a comparative composition by oral route at the same delivery dose. In some embodiments, following administration of the composition by topical route the Cmax of the substituted or unsubstituted diindolylmethane in skin increases by about 900-fold compared to the Cmax following administration of a comparative composition by oral route at the same delivery dose. In some embodiments, following administration of the composition by topical route the Cmax of the substituted or unsubstituted diindolylmethane in skin increases by about 1000-fold compared to the Cmax following administration of a comparative composition by oral route at the same delivery dose.

### Increased AUC

In some embodiments, following administration of the composition by topical route the AUC of the substituted or unsubstituted diindolylmethane in skin increases by about 1-fold to about 1000-fold compared to the AUC following administration of a comparative composition by oral route at the same delivery dose. In some embodiments, following administration of the composition by topical route the AUC of the substituted or unsubstituted diindolylmethane in skin increases by about 1-fold to about 500-fold compared to the AUC following administration of a comparative composition by oral route at the same delivery dose. In some embodiments, following administration of the composition by topical route the AUC of the substituted or unsubstituted diindolylmethane in skin increases by about 1-fold to about 100-fold compared to the AUC following administration of a comparative composition by oral route at the same delivery dose. In some embodiments, following administration of the composition by topical route the AUC of the substituted or unsubstituted diindolylmethane in skin increases by about 1-fold to about 50-fold compared to the AUC following administration of a comparative composition by oral route at the same delivery dose. In some embodiments, following administration of the composition by topical route the AUC of the substituted or unsubstituted diindolylmethane in skin increases by about 1-fold to about 20-fold compared to the AUC following administration of a comparative composition by oral route at the same delivery dose. In some embodiments, following administration of the composition by topical route the AUC of the substituted or unsubstituted diindolylmethane in skin increases by about 1-fold to about 10-fold compared to the AUC following administration of a comparative composition by oral route at the same delivery dose.

In some embodiments, following administration of the composition by topical route the AUC of the substituted or unsubstituted diindolylmethane in skin increases by about 1-fold compared to the AUC following administration of a comparative composition by oral route at the same delivery dose. In some embodiments, following administration of the composition by topical route the AUC of the substituted or unsubstituted diindolylmethane in skin increases by about 2-fold compared to the AUC following administration of a comparative composition by oral route at the same delivery dose. In some embodiments, following administration of the composition by topical route the AUC of the substituted or unsubstituted diindolylmethane in skin increases by about 3-fold compared to the AUC following administration of a comparative composition by oral route at the same delivery dose. In some embodiments, following administration of the composition by topical route the AUC of the substituted or unsubstituted diindolylmethane in skin increases by about 4-fold compared to the AUC following administration of a comparative composition by oral route at the same delivery dose. In some embodiments, following administration of the composition by topical route the AUC of the substituted or unsubstituted diindolylmethane in skin increases by about 5-fold compared to the AUC following administration of a comparative composition by oral route at the same delivery dose. In some embodiments, following administration of the composition by topical route the AUC of the substituted or unsubstituted diindolylmethane in skin increases by about 6-fold compared to the AUC following administration of a comparative composition by oral route at the same delivery dose. In some embodiments, following administration of the composition by topical route the AUC of the substituted or unsubstituted diindolylmethane in skin increases by about 7-fold compared to the AUC following administration of a comparative composition by oral route at the same delivery dose. In some embodiments, following administration of the composition by topical route the AUC of the substituted or unsubstituted diindolylmethane in skin increases by about 8-fold compared to the AUC following administration of a comparative composition by oral route at the same delivery dose. In some embodiments, following administration of the composition by topical route the AUC of the substituted or unsubstituted diindolylmethane in skin increases by about 9-fold compared to the AUC following administration of a comparative composition by oral route at the same delivery dose. In some embodiments, following administration of the composition by topical route the AUC of the substituted or unsubstituted diindolylmethane in skin increases by about 10-fold compared to the AUC following administration of a comparative composition by oral route at the same delivery dose. In some embodiments, following administration of the composition by topical route the AUC of the substituted or unsubstituted diindolylmethane in skin increases by about 20-fold compared to the AUC following administration of a comparative composition by oral route at the same delivery dose. In some embodiments, following administration of the composition by topical route the AUC of the substituted or unsubstituted diindolylmethane in skin increases by about 30-fold compared to the AUC following administration of a comparative composition by oral route at the same delivery dose. In some embodiments, following administration of the composition by topical route the AUC of the substituted or unsubstituted diindolylmethane in skin increases by about 40-fold compared to the AUC following administration of a comparative composition by oral route at the same delivery dose. In some embodiments, following administration of the composition by topical route the AUC of the substituted or unsubstituted diindolylmethane in skin increases by about 50-fold compared to the AUC following administration of a comparative composition by oral route at the same delivery dose. In some embodiments, following administration of the composition by topical route the AUC of the substituted or unsubstituted diindolylmethane in skin increases by about 60-fold compared to the AUC following administration of a comparative composition by oral route at the same delivery dose. In some embodiments, following administration of the composition by topical route the AUC of the substituted or unsubstituted diindolylmethane in skin increases by about 70-fold compared to the AUC following administration of a comparative composition by oral route at the same delivery dose. In some embodiments, following administration of the composition by topical route the AUC of the substituted or unsubstituted diindolylmethane in skin increases by about 80-fold compared to the AUC following administration of a comparative composition by oral route at the same delivery dose. In some embodiments, following administration of the composition by topical route the AUC of the substituted or unsubstituted diindolylmethane in skin increases by about 90-fold compared to the AUC following administration of a comparative composition by oral route at the same delivery dose. In some embodiments, following administration of the composition by topical route the AUC of the substituted or unsubstituted diindolylmethane in skin increases by about 100-fold compared to the AUC following administration of a comparative composition by oral route at the same delivery dose. In some embodiments, following administration of the composition by topical route the AUC of the substituted or unsubstituted diindolylmethane in skin increases by about 200-fold compared to the AUC following administration of a comparative composition by oral route at the same delivery dose. In some embodiments, following administration of the composition by topical route the AUC of the substituted or unsubstituted diindolylmethane in skin increases by about 300-fold compared to the AUC following administration of a comparative composition by oral route at the same delivery dose. In some embodiments, following administration of the composition by topical route the AUC of the substituted or unsubstituted diindolylmethane in skin increases by about 400-fold compared to the AUC following administration of a comparative composition by oral route at the same delivery dose. In some embodiments, following administration of the composition by topical route the AUC of the substituted or unsubstituted diindolylmethane in skin increases by about 500-fold compared to the AUC following administration of a comparative composition by oral route at the same delivery dose. In some embodiments, following administration of the composition by topical route the AUC of the substituted or unsubstituted diindolylmethane in skin increases by about 600-fold compared to the AUC following administration of a comparative composition by oral route at the same delivery dose. In some embodiments, following administration of the composition by topical route the AUC of the substituted or unsubstituted diindolylmethane in skin increases by about 700-fold compared to the AUC following administration of a comparative composition by oral route at the same delivery dose. In some embodiments, following administration of the composition by topical route the AUC of the substituted or unsubstituted diindolylmethane in skin increases by about 800-fold compared to the AUC following administration of a comparative composition by oral route at the same delivery dose. In some embodiments, following administration of the composition by topical route the AUC of the substituted or unsubstituted diindolylmethane in skin increases by about 800-fold compared to the AUC following administration of a comparative composition by oral route at the same delivery dose. In some embodiments, following administration of the composition by topical route the AUC of the substituted or unsubstituted diindolylmethane in skin increases by about 900-fold compared to the AUC following administration of a comparative composition by oral route at the same delivery dose. In some embodiments, following administration of the composition by topical route the AUC of the substituted or unsubstituted diindolylmethane in skin increases by about 1000-fold compared to the AUC following administration of a comparative composition by oral route at the same delivery dose.

### Decreased Tmax

In some embodiments, following administration of the composition by topical route the Tmax of the substituted or unsubstituted diindolylmethane in skin decreases by about 1-fold to about 1000-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 500-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 100-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 50-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 20-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 10-fold compared to the Tmax following administration of a comparative composition by oral route at the same delivery dose.

In some embodiments, following administration of the composition by topical route the Tmax of the substituted or unsubstituted diindolylmethane in skin decreases by about 1-fold compared to the Tmax following administration of a comparative composition by oral route at the same delivery dose. In some embodiments, following administration of the composition by topical route the Tmax of the substituted or unsubstituted diindolylmethane in skin decreases by about 2-fold compared to the Tmax following administration of a comparative composition by oral route at the same delivery dose. In some embodiments, following administration of the composition by topical route the Tmax of the substituted or unsubstituted diindolylmethane in skin decreases by about 3-fold compared to the Tmax following administration of a comparative composition by oral route at the same delivery dose. In some embodiments, following administration of the composition by topical route the Tmax of the substituted or unsubstituted diindolylmethane in skin decreases by about 4-fold compared to the Tmax following administration of a comparative composition by oral route at the same delivery dose. In some embodiments, following administration of the composition by topical route the Tmax of the substituted or unsubstituted diindolylmethane in skin decreases by about 5-fold compared to the Tmax following administration of a comparative composition by oral route at the same delivery dose. In some embodiments, following administration of the composition by topical route the Tmax of the substituted or unsubstituted diindolylmethane in skin decreases by about 6-fold compared to the Tmax following administration of a comparative composition by oral route at the same delivery dose. In some embodiments, following administration of the composition by topical route the Tmax of the substituted or unsubstituted diindolylmethane in skin decreases by about 7-fold compared to the Tmax following administration of a comparative composition by oral route at the same delivery dose. In some embodiments, following administration of the composition by topical route the Tmax of the substituted or unsubstituted diindolylmethane in skin decreases by about 8-fold compared to the Tmax following administration of a comparative composition by oral route at the same delivery dose. In some embodiments, following administration of the composition by topical route the Tmax of the substituted or unsubstituted diindolylmethane in skin decreases by about 9-fold compared to the Tmax following administration of a comparative composition by oral route at the same delivery dose. In some embodiments, following administration of the composition by topical route the Tmax of the substituted or unsubstituted diindolylmethane in skin decreases by about 10-fold compared to the Tmax following administration of a comparative composition by oral route at the same delivery dose. In some embodiments, following administration of the composition by topical route the Tmax of the substituted or unsubstituted diindolylmethane in skin decreases by about 20-fold compared to the Tmax following administration of a comparative composition by oral route at the same delivery dose. In some embodiments, following administration of the composition by topical route the Tmax of the substituted or unsubstituted diindolylmethane in skin decreases by about 30-fold compared to the Tmax following administration of a comparative composition by oral route at the same delivery dose. In some embodiments, following administration of the composition by topical route the Tmax of the substituted or unsubstituted diindolylmethane in skin decreases by about 40-fold compared to the Tmax following administration of a comparative composition by oral route at the same delivery dose. In some embodiments, following administration of the composition by topical route the Tmax of the substituted or unsubstituted diindolylmethane in skin decreases by about 50-fold compared to the Tmax following administration of a comparative composition by oral route at the same delivery dose. In some embodiments, following administration of the composition by topical route the Tmax of the substituted or unsubstituted diindolylmethane in skin decreases by about 60-fold compared to the Tmax following administration of a comparative composition by oral route at the same delivery dose. In some embodiments, following administration of the composition by topical route the Tmax of the substituted or unsubstituted diindolylmethane in skin decreases by about 70-fold compared to the Tmax following administration of a comparative composition by oral route at the same delivery dose. In some embodiments, following administration of the composition by topical route the Tmax of the substituted or unsubstituted diindolylmethane in skin decreases by about 80-fold compared to the Tmax following administration of a comparative composition by oral route at the same delivery dose. In some embodiments, following administration of the composition by topical route the Tmax of the substituted or unsubstituted diindolylmethane in skin decreases by about 100-fold compared to the Tmax following administration of a comparative composition by oral route at the same delivery dose. In some embodiments, following administration of the composition by topical route the Tmax of the substituted or unsubstituted diindolylmethane in skin decreases by about 200-fold compared to the Tmax following administration of a comparative composition by oral route at the same delivery dose. In some embodiments, following administration of the composition by topical route the Tmax of the substituted or unsubstituted diindolylmethane in skin decreases by about 300-fold compared to the Tmax following administration of a comparative composition by oral route at the same delivery dose. In some embodiments, following administration of the composition by topical route the Tmax of the substituted or unsubstituted diindolylmethane in skin decreases by about 400-fold compared to the Tmax following administration of a comparative composition by oral route at the same delivery dose. In some embodiments, following administration of the composition by topical route the Tmax of the substituted or unsubstituted diindolylmethane in skin decreases by about 500-fold compared to the Tmax following administration of a comparative composition by oral route at the same delivery dose. In some embodiments, following administration of the composition by topical route the Tmax of the substituted or unsubstituted diindolylmethane in skin decreases by about 600-fold compared to the Tmax following administration of a comparative composition by oral route at the same delivery dose. In some embodiments, following administration of the composition by topical route the Tmax of the substituted or unsubstituted diindolylmethane in skin decreases by about 700-fold compared to the Tmax following administration of a comparative composition by oral route at the same delivery dose. In some embodiments, following administration of the composition by topical route the Tmax of the substituted or unsubstituted diindolylmethane in skin decreases by about 800-fold compared to the Tmax following administration of a comparative composition by oral route at the same delivery dose. In some embodiments, following administration of the composition by topical route the Tmax of the substituted or unsubstituted diindolylmethane in skin decreases by about 900-fold compared to the Tmax following administration of a comparative composition by oral route at the same delivery dose. In some embodiments, following administration of the composition by topical route the Tmax of the substituted or unsubstituted diindolylmethane in skin decreases by about 1000-fold compared to the Tmax following administration of a comparative composition by oral route at the same delivery dose.

### DIM Composition, Formulations, Routes of Administration and Delivery Dosage

In some embodiments, the compositions described herein comprise a substituted or unsubstituted diindolylmethane. In some embodiments, the compositions described herein comprise a substituted or unsubstituted diindolylmethane that has been adapted to improve dermal bioavailability. In some embodiments, the substituted or unsubstituted diindolylmethane that has been adapted to improve dermal bioavailability is Bioresponse-diindolylmethane (BR-DIM). In some embodiments, the composition described herein comprises substituted or unsubstituted diindolylmethane that has not been adapted for improved dermal bioavailability. In some embodiments, the substituted or unsubstituted diindolylmethane that has not been adapted for improved dermal bioavailability is not BR-DIM. In some embodiments, the compositions described herein further comprise a substituted or unsubstituted retinoic acid based component.

In some embodiments, the retinoic acid based component is any such compound known in the art that is suitable for topical, oral, or topical administration. For example, in some embodiments, it is selected from a substituted or unsubstituted first generation retinoid, a substituted or unsubstituted second generation retinoid, and a substituted or unsubstituted third generation retinoid. In some embodiments, the retinoid is a substituted or unsubstituted first generation retinoid. In some embodiments, the substituted or unsubstituted first generation retinoid is selected from a substituted or unsubstituted retinol, a substituted or unsubstituted retinal, a substituted or unsubstituted tretinoin (e.g., retinoic acid or Retin A), a substituted or unsubstituted isotretinoin (e.g., Accutane™), and a substituted or unsubstituted alitretinoin. In some embodiments, the retinoid is vitamin A. In some embodiments, the retinoid is a substituted or unsubstituted second generation retinoid selected from a substituted or unsubstituted etretinate, and a substituted or unsubstituted acitretin. In some embodiments, the retinoid is a substituted or unsubstituted third generation retinoid selected from a substituted or unsubstituted tazarotene, a substituted or unsubstituted bexarotene, and a substituted or unsubstituted adapalene.

In some embodiments the composition comprising substituted or unsubstituted diindolylmethane is administered at a delivery dose that is sufficiently low to avoid toxicity, whilst still maintaining the required pharmaceutical effect. In some embodiments, the delivery dose of the composition varies depending upon whether it is a natural or synthetic product. In some embodiments, the delivery dose of the composition comprising the substituted or unsubstituted diindolylmethane is determined by whether or not the diindolylmethane has been adapted to improve dermal bioavailability. In some embodiments, the delivery dose for a composition comprising DIM is less than the delivery dose for a composition comprising substituted or unsubstituted diindolylmethane that has not been adapted to improve dermal bioavailability.

In some embodiments, the delivery dose is from about 0.01 mg to about 100 mg, from about 0.01 mg to about 75 mg, from about 0.01 mg to about 50 mg, from about 0.01 mg to about 25 mg, from about 0.01 mg to about 20 mg, from about 0.01 mg to about 15 mg, from about 0.01 mg to about 14 mg, from about 0.01 mg to about 13 mg, from about 0.01 mg to about 12 mg, from about 0.01 mg to about 11 mg, from about 0.01 mg to about 10 mg, from about 0.01 mg to about 9 mg, from about 0.01 mg to about 8 mg, from about 0.01 mg to about 7 mg, from about 0.01 mg to about 6 mg, from about 0.01 mg to about 9 mg, from about 0.01 mg to about 5 mg, from about 0.01 mg to about 4 mg, from about 0.01 mg to about 3 mg, from about 0.01 mg to about 2 mg, or from about 0.01 mg to about 1 mg.

In some embodiments, the delivery dose is from about 0.1 mg to about 100 mg, from about 0.1 mg to about 75 mg, from about 0.1 mg to about 50 mg, from about 0.1 mg to about 25 mg, from about 0.1 mg to about 20 mg, from about 0.1 mg to about 15 mg, from about 0.1 mg to about 14 mg, from about 0.1 mg to about 13 mg, from about 0.1 mg to about 12 mg, from about 0.1 mg to about 11 mg, from about 0.1 mg to about 10 mg, from about 0.1 mg to about 9 mg, from about 0.1 mg to about 8 mg, from about 0.1 mg to about 7 mg, from about 0.1 mg to about 6 mg, from about 0.1 mg to about 9 mg, from about 0.1 mg to about 5 mg, from about 0.1 mg to about 4 mg, from about 0.1 mg to about 3 mg, from about 0.1 mg to about 2 mg, or from about 0.1 mg to about 1 mg.

In some embodiments, the delivery dose is from about 0.2 mg to about 100 mg, from about 0.2 mg to about 75 mg, from about 0.2 mg to about 50 mg, from about 0.2 mg to about 25 mg, from about 0.2 mg to about 20 mg, from about 0.2 mg to about 15 mg, from about 0.2 mg to about 14 mg, from about 0.2 mg to about 13 mg, from about 0.2 mg to about 12 mg, from about 0.2 mg to about 11 mg, from about 0.2 mg to about 10 mg, from about 0.2 mg to about 9 mg, from about 0.2 mg to about 8 mg, from about 0.2 mg to about 7 mg, from about 0.2 mg to about 6 mg, from about 0.2 mg to about 9 mg, from about 0.2 mg to about 5 mg, from about 0.2 mg to about 4 mg, from about 0.2 mg to about 3 mg, from about 0.2 mg to about 2 mg, or from about 0.2 mg to about 1 mg.

In some embodiments, the delivery dose is from about 0.3 mg to about 100 mg, from about 0.3 mg to about 75 mg, from about 0.3 mg to about 50 mg, from about 0.3 mg to about 25 mg, from about 0.3 mg to about 20 mg, from about 0.3 mg to about 15 mg, from about 0.3 mg to about 14 mg, from about 0.3 mg to about 13 mg, from about 0.3 mg to about 12 mg, from about 0.3 mg to about 11 mg, from about 0.3 mg to about 10 mg, from about 0.3 mg to about 9 mg, from about 0.3 mg to about 8 mg, from about 0.3 mg to about 7 mg, from about 0.3 mg to about 6 mg, from about 0.3 mg to about 9 mg, from about 0.3 mg to about 5 mg, from about 0.3 mg to about 4 mg, from about 0.3 mg to about 3 mg, from about 0.3 mg to about 2 mg, or from about 0.3 mg to about 1 mg.

In some embodiments, the delivery dose is from about 0.4 mg to about 100 mg, from about 0.4 mg to about 75 mg, from about 0.4 mg to about 50 mg, from about 0.4 mg to about 25 mg, from about 0.4 mg to about 20 mg, from about 0.4 mg to about 15 mg, from about 0.4 mg to about 14 mg, from about 0.4 mg to about 13 mg, from about 0.4 mg to about 12 mg, from about 0.4 mg to about 11 mg, from about 0.4 mg to about 10 mg, from about 0.4 mg to about 9 mg, from about 0.4 mg to about 8 mg, from about 0.4 mg to about 7 mg, from about 0.4 mg to about 6 mg, from about 0.4 mg to about 9 mg, from about 0.4 mg to about 5 mg, from about 0.4 mg to about 4 mg, from about 0.4 mg to about 3 mg, from about 0.4 mg to about 2 mg, or from about 0.4 mg to about 1 mg.

In some embodiments, the delivery dose is from about 0.5 mg to about 100 mg, from about 0.5 mg to about 75 mg, from about 0.5 mg to about 50 mg, from about 0.5 mg to about 25 mg, from about 0.5 mg to about 20 mg, from about 0.5 mg to about 15 mg, from about 0.5 mg to about 14 mg, from about 0.5 mg to about 13 mg, from about 0.5 mg to about 12 mg, from about 0.5 mg to about 11 mg, from about 0.5 mg to about 10 mg, from about 0.5 mg to about 9 mg, from about 0.5 mg to about 8 mg, from about 0.5 mg to about 7 mg, from about 0.5 mg to about 6 mg, from about 0.5 mg to about 9 mg, from about 0.5 mg to about 5 mg, from about 0.5 mg to about 4 mg, from about 0.5 mg to about 3 mg, from about 0.5 mg to about 2 mg, or from about 0.5 mg to about 1 mg.

In some embodiments, the delivery dose of the compositions described herein provides a daily dose of the substituted or unsubstituted diindolylmethane from about 10 mg to about 20 mg, from about 15 mg to about 25 mg, from about 20 mg to about 30 mg, from about 25 mg to about 35 mg, from about 30 mg to about 40 mg, from about 35 mg to about 45 mg, from about 40 mg to about 50 mg, from about 45 mg to about 55 mg, from about 50 mg to about 100 mg, from about 55 mg to about 150 mg, from about 60 mg to about 200 mg, from about 65 mg to about 250 mg, from about 70 mg to about 300 mg.

In some embodiments, the delivery dose of the compositions described herein provides a daily dose of substituted or unsubstituted diindolylmethane that is at least 20 mg, at least 30 mg, at least 40 mg, at least 55 mg, at least 65 mg, at least 75 mg, at least 90 mg, at least 100 mg, at least 110 mg, at least 120 mg, at least 130 mg, at least 140 mg, or at least 150 mg.

In some embodiments, the composition comprising a substituted or unsubstituted diindolylmethane is administered in any of the above dosages, including the higher dosages, if desired. In some embodiments, the composition comprising a substituted or unsubstituted diindolylmethane that has been adapted to improve dermal bioavailability is administered in any of the above dosages, including the higher dosages, if desired. In some embodiments, the substituted or unsubstituted diindolylmethane that has been adapted to improve dermal bioavailability is BR-DIM.

In some embodiments, the composition comprising substituted or unsubstituted diinolylmethane is a formulation which is administered by topical route. The term “formulation which is administered by topical route” as used herein refers to a cosmetic agent delivery formulation wherein an active compound is provided for penetration into and/or absorption by tissues and cells of various skin layers, such as epidermis, dermis, and/or hypodermis. The term encompasses suitable dosage forms, including gels, creams, ointments, liquids or viscous liquids (standalone or impregnated in a substrate, matrix or sponge), transdermal patches, bandages, etc. The standalone liquids or viscous liquids can be a solution, emulsion or a suspension.

In some embodiments, a comparative composition comprising substituted or unsubstituted diinolylmethane is a formulation which is administered by oral route. In some embodiments, the formulation which is administered by oral route is in the form of a tablet, capsule, or powder.

In some embodiments, a comparative composition comprising substituted or unsubstituted diinolylmethane is a formulation which is administered by sublingual or bucaal route. In some embodiments, the formulation which is administered by oral route is in the form of a tablet, lonzenge, or film.

In some embodiments the delivery dose for topical administration is no more than 15 mg. In some embodiments the delivery dose for topical administration is no more than 14 mg. In some embodiments the delivery dose for topical administration is no more than 13 mg. In some embodiments the delivery dose for topical administration is no more than 12 mg. In some embodiments the delivery dose for topical administration is no more than 11 mg. In some embodiments the delivery dose for topical administration is no more than 10 mg. In some embodiments the delivery dose for topical administration is no more than 9 mg. In some embodiments the delivery dose for topical administration is no more than 8 mg. In some embodiments the delivery dose for topical administration is no more than 7 mg. In some embodiments the delivery dose for topical administration is no more than 6 mg. In some embodiments the delivery dose for topical administration is no more than 5 mg. In some embodiments the delivery dose for topical administration is no more than 4 mg. In some embodiments the delivery dose for topical administration is no more than 3 mg. In some embodiments the delivery dose for topical administration is no more than 2 mg. In some embodiments the delivery dose for topical administration is no more than 1 mg. In some embodiments the delivery dose for topical administration is no more than 0.5 mg.

In some embodiments the delivery dose for topical administration is about 15 mg. In some embodiments the delivery dose for topical administration is about 14 mg. In some embodiments the delivery dose for topical administration is about 13 mg. In some embodiments the delivery dose for topical administration is about 12 mg. In some embodiments the delivery dose for topical administration is about 11 mg. In some embodiments the delivery dose for topical administration is about 10 mg. In some embodiments the delivery dose for topical administration is about 9 mg. In some embodiments the delivery dose for topical administration is about 8 mg. In some embodiments the delivery dose for topical administration is about 7 mg. In some embodiments the delivery dose for topical administration is about 6 mg. In some embodiments the delivery dose for topical administration is about 5 mg. In some embodiments the delivery dose for topical administration is about 4 mg. In some embodiments the delivery dose for topical administration is about 3 mg. In some embodiments the delivery dose for topical administration is about 2 mg. In some embodiments the delivery dose for topical administration is about 1 mg. In some embodiments the delivery dose for topical administration is about 0.5 mg.

In some embodiments, the delivery doses for administration by topical route and by oral route are the same. In some embodiments, the delivery dose for administration by topical route is less than the delivery dose for administration by oral route.

In some embodiments, the compositions described herein comprise substituted or unsubstituted diindolylmethane in a dose which is a fraction of the daily dose, such as a half of the daily dose, or a quarter of the daily dose, and thus is present in a half or a quarter of any of the dosages recited above. In these embodiments, each dose fraction is taken separately over time to spread the dose across the day.

In some embodiments, the compositions described herein further comprise a substituted or unsubstituted retinoid compound (e.g. retinoic acid, vitamin C, retinyl palmitate). In some embodiments, the retinoid compound is administered at a dosage that it is sufficiently low to avoid toxicity, whilst still maintaining the required pharmaceutical effect. In some embodiments, the delivery dosage of the retinoic compound depends on the dermal bioavailability of the same. In some embodiments, the dermal bioavailability of the substituted or unsubstituted retinoic compound varies depending upon whether it is a natural or synthetic product. In some embodiments, the dermal bioavailability of the substituted or unsubstituted retinoic compound may vary depending on whether it has been adapted to improve its dermal bioavailability.

In some embodiments, daily dosage of the composition comprising a substituted or unsubstituted diindolylmethane and optionally a substituted or unsubstituted retinoic compound is provided in the form of one or more unit doses In some embodiments, daily dosage of the composition comprising a substituted or unsubstituted diindolylmethane and optionally a substituted or unsubstituted retinoic acid based component is provided in the form of 2 to 4 unit doses. In these embodiments the two or more unit doses are taken during the course of a single day, such as one unit dose in the morning and one unit dose in the evening, or four unit doses spread evenly across the day, or two unit doses simultaneously twice a day.

### Methods for Preparation

In some embodiments, are provided, methods for preparing the compositions described herein, for use in the methods described herein. In some embodiments, using the methods described herein, a composition suitable for topical administration is prepared.

In some embodiments, any methods known in the art for blending or mixing various components of the composition are employed. In some embodiments, the methods employed are methods for blending and/or mixing powders or fluids. In some embodiments, the method comprises mixing substituted or unsubstituted diindolylmethane with one or more pharmaceutically acceptable excipients and/or additives, to form the composition. In some embodiments, the substituted or unsubstituted diindolylmethane and the one or more pharmaceutically acceptable excipients and/or additives are each, separately from each other, mixed with one or more pharmaceutically acceptable excipients and/or additives before being mixed together to form the composition. In some embodiments, the substituted or unsubstituted diindolylmethane and the pharmaceutically acceptable excipients are added sequentially to the mixture during the mixing process.

In some embodiments, the selection of the pharmaceutically acceptable excipients and the method of blending are adapted in order to overcome any mixing, flow and fill issues or punch issues with the composition. In some embodiments, the composition comprising a substituted or unsubstituted diindolylmethane is provided in micro-encapsulated form, such that the powder particles have a tendency to clump together. In some embodiments, the composition comprising a first component, comprising a substituted or unsubstituted diindolylmethane is blended or mixed using a method that is adapted to avoid creating hot spots of increased concentrations of the active ingredients.

### NON-LIMITING EMBODIMENTS

The following specific, non-limiting embodiments are to be construed as merely illustrative, and do not limit the present disclosure of the scope of the disclosure. Without further elaboration, it is believed that one skilled in the art can, based on the description herein, utilize the present disclosure to its fullest extent.

1. A topical composition for cosmetic use in a subject in need thereof, wherein the composition comprises substituted or unsubstituted diindolylmethane that is optionally emulsified, and a pharmaceutically acceptable carrier, and wherein the composition does not cause irritation or allergic reaction after topical application.

2. A topical composition for cosmetic use in a subject in need thereof, wherein the composition comprises from about 0.1 wt % to about 10 wt % of substituted or unsubstituted diindolylmethane and a pharmaceutically acceptable carrier, and wherein the composition does not cause irritation or allergic reaction after topical application.

3. A topical composition for cosmetic use in a subject in need thereof, wherein the composition comprises from about 0.1 wt % to about 10 wt % of emulsified substituted or unsubstituted diindolylmethane and a pharmaceutically acceptable carrier, and wherein the composition does not cause irritation or allergic reaction after topical application.

4. The topical composition of any one of embodiments 1-3, wherein the administration by topical route increases the dermal bioavailability of the substituted or unsubstituted diindolylmethane by about 1-fold to about 1000-fold compared to the dermal bioavailability of the substituted or unsubstituted diindolylmethane of a comparative composition administered by oral route at the same delivery dose.

5. The topical composition of any one of embodiments 1-3, wherein the administration by topical route increases the dermal bioavailability of the substituted or unsubstituted diindolylmethane by about 1-fold compared to the dermal bioavailability of the substituted or unsubstituted diindolylmethane of a comparative composition administered by oral route at the same delivery dose.

6. The topical composition of any one of embodiments 1-3, wherein the administration by topical route increases the dermal bioavailability of the substituted or unsubstituted diindolylmethane by about 2-fold compared to the dermal bioavailability of the substituted or unsubstituted diindolylmethane of a comparative composition administered by oral route at the same delivery dose.

7. The topical composition of any one of embodiments 1-3, wherein the administration by topical route increases the dermal bioavailability of the substituted or unsubstituted diindolylmethane by about 3-fold compared to the dermal bioavailability of the substituted or unsubstituted diindolylmethane of a comparative composition administered by oral route at the same delivery dose.

8. The topical composition of any one of embodiment 1-3, wherein the administration by topical route increases the dermal bioavailability of the substituted or unsubstituted diindolylmethane by about 4-fold compared to the dermal bioavailability of the substituted or unsubstituted diindolylmethane of a comparative composition administered by oral route at the same delivery dose.

9. The topical composition of any one of embodiments 1-3, wherein the administration by topical route increases the dermal bioavailability of the substituted or unsubstituted diindolylmethane by about 5-fold compared to the dermal bioavailability of the substituted or unsubstituted diindolylmethane of a comparative composition administered by oral route at the same delivery dose.

10. The topical composition of any one of embodiments 1-3, wherein the administration by topical route increases the dermal bioavailability of the substituted or unsubstituted diindolylmethane by about 10-fold compared to the dermal bioavailability of the substituted or unsubstituted diindolylmethane of a comparative composition administered by oral route at the same delivery dose.

11. The topical composition of any one of embodiments 1-3, wherein the administration by topical route increases the dermal bioavailability of the substituted or unsubstituted diindolylmethane by about 20-fold compared to the dermal bioavailability of the substituted or unsubstituted diindolylmethane of a comparative composition administered by oral route at the same delivery dose.

12. The topical composition of any one of embodiments 1-3, wherein the administration by topical route increases the dermal bioavailability of the substituted or unsubstituted diindolylmethane by about 30-fold compared to the dermal bioavailability of the substituted or unsubstituted diindolylmethane of a comparative composition administered by oral route at the same delivery dose.

13. The topical composition of any one of embodiments 1-3, wherein the administration by topical route increases the dermal bioavailability of the substituted or unsubstituted diindolylmethane by about 40-fold compared to the dermal bioavailability of the substituted or unsubstituted diindolylmethane of a comparative composition administered by oral route at the same delivery dose.

14. The topical composition of any one of embodiments 1-3, wherein the administration by topical route increases the dermal bioavailability of the substituted or unsubstituted diindolylmethane by about 50-fold compared to the dermal bioavailability of the substituted or unsubstituted diindolylmethane of a comparative composition administered by oral route at the same delivery dose.

15. The topical composition of any one of embodiments 1-3, wherein the administration by topical route increases the dermal bioavailability of the substituted or unsubstituted diindolylmethane by about 60-fold compared to the dermal bioavailability of the substituted or unsubstituted diindolylmethane of a comparative composition administered by oral route at the same delivery dose.

16. The topical composition of any one of embodiments 1-3, wherein the administration by topical route increases the dermal bioavailability of the substituted or unsubstituted diindolylmethane by about 70-fold compared to the dermal bioavailability of the substituted or unsubstituted diindolylmethane of a comparative composition administered by oral route at the same delivery dose.

17. The topical composition of any one of embodiments 1-3, wherein the administration by topical route increases the dermal bioavailability of the substituted or unsubstituted diindolylmethane by about 80-fold compared to the dermal bioavailability of the substituted or unsubstituted diindolylmethane of a comparative composition administered by oral route at the same delivery dose.

18. The topical composition of any one of embodiments 1-3, wherein the administration by topical route increases the dermal bioavailability of the substituted or unsubstituted diindolylmethane by about 100-fold compared to the dermal bioavailability of the substituted or unsubstituted diindolylmethane of a comparative composition administered by oral route at the same delivery dose.

19. The topical composition of any one of embodiments 1-3, wherein the administration by topical route increases the dermal bioavailability of the substituted or unsubstituted diindolylmethane by about 200-fold compared to the dermal bioavailability of the substituted or unsubstituted diindolylmethane of a comparative composition administered by oral route at the same delivery dose.

20. The topical composition of any one of embodiments 1-3, wherein the administration by topical route increases the dermal bioavailability of the substituted or unsubstituted diindolylmethane by about 300-fold compared to the dermal bioavailability of the substituted or unsubstituted diindolylmethane of a comparative composition administered by oral route at the same delivery dose.

21. The topical composition of any one of embodiments 1-3, wherein the administration by topical route increases the dermal bioavailability of the substituted or unsubstituted diindolylmethane by about 400-fold compared to the dermal bioavailability of the substituted or unsubstituted diindolylmethane of a comparative composition administered by oral route at the same delivery dose.

22. The topical composition of any one of embodiments 1-3, wherein the administration by topical route increases the dermal bioavailability of the substituted or unsubstituted diindolylmethane by about 500-fold compared to the dermal bioavailability of the substituted or unsubstituted diindolylmethane of a comparative composition administered by oral route at the same delivery dose.

23. The topical composition of any one of embodiments 1-3, wherein the administration by topical route increases the dermal bioavailability of the substituted or unsubstituted diindolylmethane by about 600-fold compared to the dermal bioavailability of the substituted or unsubstituted diindolylmethane of a comparative composition administered by oral route at the same delivery dose.

24. The topical composition of any one of embodiments 1-3, wherein the administration by topical route increases the dermal bioavailability of the substituted or unsubstituted diindolylmethane by about 700-fold compared to the dermal bioavailability of the substituted or unsubstituted diindolylmethane of a comparative composition administered by oral route at the same delivery dose.

25. The topical composition of any one of embodiments 1-3, wherein the administration by topical route increases the dermal bioavailability of the substituted or unsubstituted diindolylmethane by about 800-fold compared to the dermal bioavailability of the substituted or unsubstituted diindolylmethane of a comparative composition administered by oral route at the same delivery dose.

26. The topical composition of any one of embodiments 1-3, wherein the administration by topical route increases the dermal bioavailability of the substituted or unsubstituted diindolylmethane by about 900-fold compared to the dermal bioavailability of the substituted or unsubstituted diindolylmethane of a comparative composition administered by oral route at the same delivery dose.

27. The topical composition of any one of embodiments 1-3, wherein the administration by topical route increases the dermal bioavailability of the substituted or unsubstituted diindolylmethane by about 1000-fold compared to the dermal bioavailability of the substituted or unsubstituted diindolylmethane of a comparative composition administered by oral route at the same delivery dose.

28. The topical composition of any one of embodiments 4-27, wherein the dermal bioavailability of the substituted or unsubstituted diindolylmethane is measured following administration of a dose comprising less than 15 mg, preferably less than 10 mg, more preferably less than 5 mg of the substituted or unsubstituted diindolylmethane.

29. The topical composition of any one of embodiments 1-28, wherein following administration of the composition by topical route the Cmax of the substituted or unsubstituted diindolylmethane in skin increases by about 1-fold to about 1000-fold compared to the Cmax following administration of a comparative composition by oral route at the same delivery dose.

30. The topical composition of any one of embodiments 1-28, wherein following administration of the composition by topical route the Cmax of the substituted or unsubstituted diindolylmethane in skin increases by about 2-fold compared to the Cmax following administration of a comparative composition by oral route at the same delivery dose.

31. The topical composition of any one of embodiments 1-28, wherein following administration of the composition by topical route the Cmax of the substituted or unsubstituted diindolylmethane in skin increases by about 3-fold compared to the Cmax following administration of a comparative composition by oral route at the same delivery dose.

32. The topical composition of any one of embodiments 1-28, wherein following administration of the composition by topical route the Cmax of the substituted or unsubstituted diindolylmethane in skin increases by about 4-fold compared to the Cmax following administration of a comparative composition by oral route at the same delivery dose.

33. The topical composition of any one of embodiments 1-28, wherein following administration of the composition by topical route the Cmax of the substituted or unsubstituted diindolylmethane in skin increases by about 5-fold compared to the Cmax following administration of a comparative composition by oral route at the same delivery dose.

34. The topical composition of any one of embodiments 1-28, wherein following administration of the composition by topical route the Cmax of the substituted or unsubstituted diindolylmethane in skin increases by about 10-fold compared to the Cmax following administration of a comparative composition by oral route at the same delivery dose.

35. The topical composition of any one of embodiments 1-28, wherein following administration of the composition by topical route the Cmax of the substituted or unsubstituted diindolylmethane in skin increases by about 20-fold compared to the Cmax following administration of a comparative composition by oral route at the same delivery dose.

36. The topical composition of any one of embodiments 1-28, wherein following administration of the composition by topical route the Cmax of the substituted or unsubstituted diindolylmethane in skin increases by about 30-fold compared to the Cmax following administration of a comparative composition by oral route at the same delivery dose.

37. The topical composition of any one of embodiments 1-28, wherein following administration of the composition by topical route the Cmax of the substituted or unsubstituted diindolylmethane in skin increases by about 40-fold compared to the Cmax following administration of a comparative composition by oral route at the same delivery dose.

38. The topical composition of any one of embodiments 1-28, wherein following administration of the composition by topical route the Cmax of the substituted or unsubstituted diindolylmethane in skin increases by about 50-fold compared to the Cmax following administration of a comparative composition by oral route at the same delivery dose.

39. The topical composition of any one of embodiments 1-28, wherein following administration of the composition by topical route the Cmax of the substituted or unsubstituted diindolylmethane in skin increases by about 60-fold compared to the Cmax following administration of a comparative composition by oral route at the same delivery dose.

40. The topical composition of any one of embodiments 1-28, wherein following administration of the composition by topical route the Cmax of the substituted or unsubstituted diindolylmethane in skin increases by about 70-fold compared to the Cmax following administration of a comparative composition by oral route at the same delivery dose.

41. The topical composition of any one of embodiments 1-28, wherein following administration of the composition by topical route the Cmax of the substituted or unsubstituted diindolylmethane in skin increases by about 80-fold compared to the Cmax following administration of a comparative composition by oral route at the same delivery dose.

42. The topical composition of any one of embodiments 1-28, wherein following administration of the composition by topical route the Cmax of the substituted or unsubstituted diindolylmethane in skin increases by about 90-fold compared to the Cmax following administration of a comparative composition by oral route at the same delivery dose.

43. The topical composition of any one of embodiments 1-28, wherein following administration of the composition by topical route the Cmax of the substituted or unsubstituted diindolylmethane in skin increases by about 100-fold compared to the Cmax following administration of a comparative composition by oral route

44. The topical composition of any one of embodiments 1-28, wherein following administration of the composition by topical route the Cmax of the substituted or unsubstituted diindolylmethane in skin increases by about 200-fold compared to the Cmax following administration of a comparative composition by oral route at the same delivery dose.

45. The topical composition of any one of embodiments 1-28, wherein following administration of the composition by topical route the Cmax of the substituted or unsubstituted diindolylmethane in skin increases by about 300-fold compared to the Cmax following administration of a comparative composition by oral route at the same delivery dose.

46. The topical composition of any one of embodiments 1-28, wherein following administration of the composition by topical route the Cmax of the substituted or unsubstituted diindolylmethane in skin increases by about 400-fold compared to the Cmax following administration of a comparative composition by oral route at the same delivery dose.

47. The topical composition of any one of embodiments 1-28, wherein following administration of the composition by topical route the Cmax of the substituted or unsubstituted diindolylmethane in skin increases by about 500-fold compared to the Cmax following administration of a comparative composition by oral route at the same delivery dose.

48. The topical composition of any one of embodiments 1-28, wherein following administration of the composition by topical route the Cmax of the substituted or unsubstituted diindolylmethane in skin increases by about 600-fold compared to the Cmax following administration of a comparative composition by oral route at the same delivery dose.

49. The topical composition of any one of embodiments 1-28, wherein following administration of the composition by topical route the Cmax of the substituted or unsubstituted diindolylmethane in skin increases by about 700-fold compared to the Cmax following administration of a comparative composition by oral route at the same delivery dose.

50. The topical composition of any one of embodiments 1-28, wherein following administration of the composition by topical route the Cmax of the substituted or unsubstituted diindolylmethane in skin increases by about 800-fold compared to the Cmax following administration of a comparative composition by oral route at the same delivery dose.

51. The topical composition of any one of embodiments 1-28, wherein following administration of the composition by topical route the Cmax of the substituted or unsubstituted diindolylmethane in skin increases by about 900-fold compared to the Cmax following administration of a comparative composition by oral route at the same delivery dose.

52. The topical composition of any one of embodiments 1-28, wherein following administration of the composition by topical route the Cmax of the substituted or unsubstituted diindolylmethane in skin increases by about 1000-fold compared to the Cmax following administration of a comparative composition by oral route at the same delivery dose.

53. The topical composition of any one of embodiments 29-52, wherein the Cmax of the substituted or unsubstituted diindolylmethane in skin is measured following administration of a dose comprising less than 15 mg, preferably less than 10 mg, more preferably less than 5 mg of the substituted or unsubstituted diindolylmethane.

54. The topical composition of any one of embodiments 1-53, wherein following administration of the composition by topical route the AUC of the substituted or unsubstituted diindolylmethane in skin increases by about 1-fold to about 1000-fold compared to the AUC following administration of a comparative composition by oral route at the same delivery dose.

55. The topical composition of any one of embodiments 1-53, wherein following administration of the composition by topical route the AUC of the substituted or unsubstituted diindolylmethane in skin increases by about 2-fold compared to the AUC following administration of a comparative composition by oral route at the same delivery dose.

56. The topical composition of any one of embodiments 1-53, wherein following administration of the composition by topical route the AUC of the substituted or unsubstituted diindolylmethane in skin increases by about 3-fold compared to the AUC following administration of a comparative composition by oral route at the same delivery dose.

57. The topical composition of any one of embodiments 1-53, wherein following administration of the composition by topical route the AUC of the substituted or unsubstituted diindolylmethane in skin increases by about 4-fold compared to the AUC following administration of a comparative composition by oral route at the same delivery dose.

58. The topical composition of any one of embodiments 1-53, wherein following administration of the composition by topical route the AUC of the substituted or unsubstituted diindolylmethane in skin increases by about 5-fold compared to the AUC following administration of a comparative composition by oral route at the same delivery dose.

59. The topical composition of any one of embodiments 1-53, wherein following administration of the composition by topical route the AUC of the substituted or unsubstituted diindolylmethane in skin increases by about 6-fold compared to the AUC following administration of a comparative composition by oral route at the same delivery dose.

60. The topical composition of any one of embodiments 1-53, wherein following administration of the composition by topical route the AUC of the substituted or unsubstituted diindolylmethane in skin increases by about 7-fold compared to the AUC following administration of a comparative composition by oral route at the same delivery dose.

61. The topical composition of any one of embodiments 1-53, wherein following administration of the composition by topical route the AUC of the substituted or unsubstituted diindolylmethane in skin increases by about 8-fold compared to the AUC following administration of a comparative composition by oral route at the same delivery dose.

62. The topical composition of any one of embodiments 1-53, wherein following administration of the composition by topical route the AUC of the substituted or unsubstituted diindolylmethane in skin increases by about 9-fold compared to the AUC following administration of a comparative composition by oral route at the same delivery dose.

63. The topical composition of any one of embodiments 1-53, wherein following administration of the composition by topical route the AUC of the substituted or unsubstituted diindolylmethane in skin increases by about 10-fold compared to the AUC following administration of a comparative composition by oral route at the same delivery dose.

64. The topical composition of any one of embodiments 1-53, wherein following administration of the composition by topical route the AUC of the substituted or unsubstituted diindolylmethane in skin increases by about 20-fold compared to the AUC following administration of a comparative composition by oral route at the same delivery dose.

65. The topical composition of any one of embodiments 1-53, wherein following administration of the composition by topical route the AUC of the substituted or unsubstituted diindolylmethane in skin increases by about 30-fold compared to the AUC following administration of a comparative composition by oral route at the same delivery dose.

66. The topical composition of any one of embodiments 1-53, wherein following administration of the composition by topical route the AUC of the substituted or unsubstituted diindolylmethane in skin increases by about 40-fold compared to the AUC following administration of a comparative composition by oral route at the same delivery dose.

67. The topical composition of any one of embodiments 1-53, wherein following administration of the composition by topical route the AUC of the substituted or unsubstituted diindolylmethane in skin increases by about 50-fold compared to the AUC following administration of a comparative composition by oral route at the same delivery dose.

68. The topical composition of any one of embodiments 1-53, wherein following administration of the composition by topical route the AUC of the substituted or unsubstituted diindolylmethane in skin increases by about 60-fold compared to the AUC following administration of a comparative composition by oral route at the same delivery dose.

69. The topical composition of any one of embodiments 1-53, wherein following administration of the composition by topical route the AUC of the substituted or unsubstituted diindolylmethane in skin increases by about 70-fold compared to the AUC following administration of a comparative composition by oral route at the same delivery dose.

70. The topical composition of any one of embodiments 1-53, wherein following administration of the composition by topical route the AUC of the substituted or unsubstituted diindolylmethane in skin increases by about 80-fold compared to the AUC following administration of a comparative composition by oral route

71. The topical composition of any one of embodiments 1-53, wherein following administration of the composition by topical route the AUC of the substituted or unsubstituted diindolylmethane in skin increases by about 90-fold compared to the AUC following administration of a comparative composition by oral route at the same delivery dose.

72. The topical composition of any one of embodiments 1-53, wherein following administration of the composition by topical route the AUC of the substituted or unsubstituted diindolylmethane in skin increases by about 100-fold compared to the AUC following administration of a comparative composition by oral route at the same delivery dose.

73. The topical composition of any one of embodiments 1-53, wherein following administration of the composition by topical route the AUC of the substituted or unsubstituted diindolylmethane in skin increases by about 200-fold compared to the AUC following administration of a comparative composition by oral route at the same delivery dose.

74. The topical composition of any one of embodiments 1-53, wherein following administration of the composition by topical route the AUC of the substituted or unsubstituted diindolylmethane in skin increases by about 300-fold compared to the AUC following administration of a comparative composition by oral route at the same delivery dose.

75. The topical composition of any one of embodiments 1-53, wherein following administration of the composition by topical route the AUC of the substituted or unsubstituted diindolylmethane in skin increases by about 400-fold compared to the AUC following administration of a comparative composition by oral route at the same delivery dose.

76. The topical composition of any one of embodiments 1-53, wherein following administration of the composition by topical route the AUC of the substituted or unsubstituted diindolylmethane in skin increases by about 500-fold compared to the AUC following administration of a comparative composition by oral route at the same delivery dose.

77. The topical composition of any one of embodiments 1-53, wherein following administration of the composition by topical route the AUC of the substituted or unsubstituted diindolylmethane in skin increases by about 600-fold compared to the AUC following administration of a comparative composition by oral route at the same delivery dose.

78. The topical composition of any one of embodiments 1-53, wherein following administration of the composition by topical route the AUC of the substituted or unsubstituted diindolylmethane in skin increases by about 700-fold compared to the AUC following administration of a comparative composition by oral route at the same delivery dose.

79. The topical composition of any one of embodiments 1-53, wherein following administration of the composition by topical route the AUC of the substituted or unsubstituted diindolylmethane in skin increases by about 800-fold compared to the AUC following administration of a comparative composition by oral route

80. The topical composition of any one of embodiments 1-53, wherein following administration of the composition by topical route the AUC of the substituted or unsubstituted diindolylmethane in skin increases by about 900-fold compared to the AUC following administration of a comparative composition by oral route at the same delivery dose.

81. The topical composition of any one of embodiments 1-53, wherein following administration of the composition by topical route the AUC of the substituted or unsubstituted diindolylmethane in skin increases by about 1000-fold compared to the AUC following administration of a comparative composition by oral route at the same delivery dose.

82. The topical composition of any one of embodiments 54-81, wherein the AUC of the substituted or unsubstituted diindolylmethane in skin is measured following administration of a dose comprising less than 15 mg, preferably less than 10 mg, more preferably less than 5 mg of the substituted or unsubstituted diindolylmethane.

83. The topical composition of any one of embodiments 1-82, wherein following administration of the composition by topical route the Tmax of the substituted or unsubstituted diindolylmethane in skin decreases by about 1-fold to about 1000-fold compared to the Tmax following administration of a comparative composition by oral route at the same delivery dose.

84. The topical composition of any one of embodiments 1-82, wherein following administration of the composition by topical route the Tmax of the substituted or unsubstituted diindolylmethane in skin decreases by about 1-fold compared to the Tmax following administration of a comparative composition by oral route at the same delivery dose.

85. The topical composition of any one of embodiments 1-82, wherein following administration of the composition by topical route the Tmax of the substituted or unsubstituted diindolylmethane in skin decreases by about 2-fold compared to the Tmax following administration of a comparative composition by oral route at the same delivery dose.

86. The topical composition of any one of embodiments 1-82, wherein following administration of the composition by topical route the Tmax of the substituted or unsubstituted diindolylmethane in skin decreases by about 3-fold compared to the Tmax following administration of a comparative composition by oral route at the same delivery dose.

87. The topical composition of any one of embodiments 1-82, wherein following administration of the composition by topical route the Tmax of the substituted or unsubstituted diindolylmethane in skin decreases by about 4-fold compared to the Tmax following administration of a comparative composition by oral route at the same delivery dose.

88. The topical composition of any one of embodiments 1-82, wherein following administration of the composition by topical route the Tmax of the substituted or unsubstituted diindolylmethane in skin decreases by about 5-fold compared to the Tmax following administration of a comparative composition by oral route at the same delivery dose.

89. The topical composition of any one of embodiments 1-82, wherein following administration of the composition by topical route the Tmax of the substituted or unsubstituted diindolylmethane in skin decreases by about 10-fold compared to the Tmax following administration of a comparative composition by oral route at the same delivery dose.

90. The topical composition of any one of embodiments 1-82, wherein following administration of the composition by topical route the Tmax of the substituted or unsubstituted diindolylmethane in skin decreases by about 20-fold compared to the Tmax following administration of a comparative composition by oral route at the same delivery dose.

91. The topical composition of any one of embodiments 1-82, wherein following administration of the composition by topical route the Tmax of the substituted or unsubstituted diindolylmethane in skin decreases by about 30-fold compared to the Tmax following administration of a comparative composition by oral route at the same delivery dose.

92. The topical composition of any one of embodiments 1-82, wherein following administration of the composition by topical route the Tmax of the substituted or unsubstituted diindolylmethane in skin decreases by about 40-fold compared to the Tmax following administration of a comparative composition by oral route at the same delivery dose.

93. The topical composition of any one of embodiments 1-82, wherein following administration of the composition by topical route the Tmax of the substituted or unsubstituted diindolylmethane in skin decreases by about 50-fold compared to the Tmax following administration of a comparative composition by oral route at the same delivery dose.

94. The topical composition of any one of embodiments 1-82, wherein following administration of the composition by topical route the Tmax of the substituted or unsubstituted diindolylmethane in skin decreases by about 60-fold compared to the Tmax following administration of a comparative composition by oral route at the same delivery dose.

95. The topical composition of any one of embodiments 1-82, wherein following administration of the composition by topical route the Tmax of the substituted or unsubstituted diindolylmethane in skin decreases by about 70-fold compared to the Tmax following administration of a comparative composition by oral route at the same delivery dose.

96. The topical composition of any one of embodiments 1-82, wherein following administration of the composition by topical route the Tmax of the substituted or unsubstituted diindolylmethane in skin decreases by about 80-fold compared to the Tmax following administration of a comparative composition by oral route at the same delivery dose.

97. The topical composition of any one of embodiments 1-82, wherein following administration of the composition by topical route the Tmax of the substituted or unsubstituted diindolylmethane in skin decreases by about 90-fold compared to the Tmax following administration of a comparative composition by oral route

98. The topical composition of any one of embodiments 1-82, wherein following administration of the composition by topical route the Tmax of the substituted or unsubstituted diindolylmethane in skin decreases by about 100-fold compared to the Tmax following administration of a comparative composition by oral route at the same delivery dose.

99. The topical composition of any one of embodiments 1-82, wherein following administration of the composition by topical route the Tmax of the substituted or unsubstituted diindolylmethane in skin decreases by about 200-fold compared to the Tmax following administration of a comparative composition by oral route at the same delivery dose.

100. The topical composition of any one of embodiments 1-82, wherein following administration of the composition by topical route the Tmax of the substituted or unsubstituted diindolylmethane in skin decreases by about 300-fold compared to the Tmax following administration of a comparative composition by oral route at the same delivery dose.

101. The topical composition of any one of embodiments 1-82, wherein following administration of the composition by topical route the Tmax of the substituted or unsubstituted diindolylmethane in skin decreases by about 400-fold compared to the Tmax following administration of a comparative composition by oral route at the same delivery dose.

102. The topical composition of any one of embodiments 1-82, wherein following administration of the composition by topical route the Tmax of the substituted or unsubstituted diindolylmethane in skin decreases by about 500-fold compared to the Tmax following administration of a comparative composition by oral route at the same delivery dose.

103. The topical composition of any one of embodiments 1-82, wherein following administration of the composition by topical route the Tmax of the substituted or unsubstituted diindolylmethane in skin decreases by about 600-fold compared to the Tmax following administration of a comparative composition by oral route at the same delivery dose.

104. The topical composition of any one of embodiments 1-82, wherein following administration of the composition by topical route the Tmax of the substituted or unsubstituted diindolylmethane in skin decreases by about 700-fold compared to the Tmax following administration of a comparative composition by oral route at the same delivery dose.

105. The topical composition of any one of embodiments 1-82, wherein following administration of the composition by topical route the Tmax of the substituted or unsubstituted diindolylmethane in skin decreases by about 800-fold compared to the Tmax following administration of a comparative composition by oral route at the same delivery dose.

106. The topical composition of any one of embodiments 1-82, wherein following administration of the composition by topical route the Tmax of the substituted or unsubstituted diindolylmethane in skin decreases by about 900-fold compared to the Tmax following administration of a comparative composition by oral route

107. The topical composition of any one of embodiments 1-82, wherein following administration of the composition by topical route the Tmax of the substituted or unsubstituted diindolylmethane in skin decreases by about 1000-fold compared to the Tmax following administration of a comparative composition by oral route at the same delivery dose.

108. The topical composition of any one of embodiments 83-107, wherein the Tmax of the substituted or unsubstituted diindolylmethane in skin is measured following administration of a dose comprising less than 15mg, preferably less than 10 mg, more preferably less than 5 mg of the substituted or unsubstituted diindolylmethane.

109. The topical composition of any one of embodiments 1-108, wherein the composition comprises from about 0.1 wt % to about 8.0 wt % of substituted or unsubstituted diindolylmethane.

110. The topical composition of any one of embodiments 1-108, wherein the composition comprises from about 0.1 wt % to about 5.0 wt % of substituted or unsubstituted diindolylmethane.

111. The topical composition of any one of embodiments 1-108, wherein the composition comprises from about 0.1 wt % to about 4.0 wt % of substituted or unsubstituted diindolylmethane.

112. The topical composition of any one of embodiments 1-108, wherein the composition comprises from about 0.1 wt % to about 3.0 wt % of substituted or unsubstituted diindolylmethane.

113. The topical composition of any one of embodiments 1-108, wherein the composition comprises from about 0.2 wt % to about 3.0 wt % of substituted or unsubstituted diindolylmethane.

114. The topical composition of any one of embodiments 1-108, wherein the composition comprises from about 0.3 wt % to about 3.0 wt % of substituted or unsubstituted diindolylmethane.

115. The topical composition of any one of embodiments 1-108, wherein the composition comprises from about 0.5 wt % to about 2.5 wt % of substituted or unsubstituted diindolylmethane.

116. The topical composition of any one of embodiments 1-108, wherein the composition comprises from about 0.3 wt % to about 0.8 wt % of substituted or unsubstituted diindolylmethane.

117. The topical composition of any one of embodiments 1-108, wherein the composition comprises about 0.5 wt % of substituted or unsubstituted diindolylmethane.

118. The topical composition of any one of embodiments 1-108, wherein the composition comprises from about 0.8 wt % to about 1.2 wt % of substituted or unsubstituted diindolylmethane.

119. The topical composition of any one of embodiments 1-108, wherein the composition comprises from about 1.0 wt % of substituted or unsubstituted diindolylmethane.

120. The topical composition of any one of embodiments 1-108, wherein the composition comprises from about 1.2 wt % to about 2.0 wt % of substituted or unsubstituted diindolylmethane.

121. The topical composition of any one of embodiments 1-108, wherein the composition comprises about 1.5 wt % of substituted or unsubstituted diindolylmethane.

122. The topical composition of any one of embodiments 1-108, wherein the composition comprises from about 2.0 wt % to about 3.0 wt % of substituted or unsubstituted diindolylmethane.

123. The topical composition of any one of embodiments 1-108, wherein the composition comprises about 2.5 wt % of substituted or unsubstituted diindolylmethane.

124. The topical composition of any one of embodiments 1-123, wherein the substituted or unsubstituted diindolylmethane is unsubstituted diindolylmethane.

125. The topical composition of any one of embodiments 1-123, wherein the substituted or unsubstituted diindolylmethane is unsubstituted 3,3′-diindolylmethane.

126. The topical composition of any one of embodiments 1-125, wherein the composition further comprises a retinoid compound.

127. The topical composition of embodiment 126, wherein the retinoid compound is a substituted or unsubstituted retinoic acid.

128. The topical composition of embodiment 126, wherein the retinoid compound is vitamin A.

129. The topical composition of embodiment 126, wherein the retinoid compound is retinyl palmitate.

130. The topical composition of any one of embodiments 1-129, wherein the composition is essentially free of any retinoid compound.

131. The topical composition of any one of embodiments 1-129, wherein the composition is free of any retinoid compound.

132. The topical composition of any one of embodiments 1-129, wherein the composition is essentially free of any cosmetically active agent other than the substituted or unsubstituted diindolylmethane.

133. The topical composition of any one of embodiments 1-129, wherein the composition is free of any cosmetically active agent other than the substituted or unsubstituted diindolylmethane.

134. The topical composition of any one of embodiments 1-133, wherein the cosmetic use comprises supporting skin integrity.

135. The topical composition of any one of embodiments 1-134, wherein the cosmetic use comprises enhancing skin appearance.

136. The topical composition of any one of embodiments 1-135, wherein the cosmetic use comprises relieving skin stress.

137. The topical composition of any one of embodiments 1-136, wherein the cosmetic use comprises slowing down skin aging.

138. The topical composition of any one of embodiments 1-137, wherein the cosmetic use comprises reducing the number and/or severity of wrinkles.

139. The topical composition of any one of embodiments 1-13, wherein the cosmetic use comprises reducing the number and/or severity of fine lines.

140. The topical composition of any one of embodiments 1-139, wherein the cosmetic use comprises reducing skin laxity.

141. The topical composition of any one of embodiments 1-140, wherein the cosmetic use comprises reducing the number, area, and/or darkness of age spots.

142. The topical composition of any one of embodiments 1-141, wherein the cosmetic use comprises reducing the number, area, and/or darkness of stretch marks.

143. The topical composition of any one of embodiments 1-142, wherein the cosmetic use comprises reducing the number, area, and/or darkness of spider veins.

144. The topical composition of any one of embodiments 1-143, wherein the cosmetic use comprises reducing skin dryness.

145. The topical composition of any one of embodiments 1-144, wherein the cosmetic use comprises reducing skin oil buildup.

146. The topical composition of any one of embodiments 1-145, wherein the cosmetic use comprises repairing sun damage.

147. The topical composition of any one of embodiments 1-146, wherein the cosmetic use comprises alleviating peeling skin.

148. The topical composition of any one of embodiments 1-147, wherein the cosmetic use comprises alleviating scaling skin.

149. The topical composition of any one of embodiments 1-148, wherein the cosmetic use comprises improving skin tone.

150. The topical composition of any one of embodiments 1-149, wherein the cosmetic use comprises reducing skin dullness.

151. The topical composition of any one of embodiments 1-150, wherein the cosmetic use comprises reducing appearance of mole.

152. The topical composition of any one of embodiments 1-151, wherein the cosmetic use comprises reducing appearance of scar.

153. The topical composition of any one of embodiments 1-152, wherein the cosmetic use comprises reducing appearance of birthmark.

154. The topical composition of any one of embodiments 1-153, wherein the cosmetic use comprises reducing skin blushing or skin flushing.

155. The topical composition of any one of embodiments 1-154, wherein the cosmetic use comprises reducing under-eye dark circle.

156. The topical composition of any one of embodiments 1-155, wherein the cosmetic use comprises reducing under-eye bag.

157. The topical composition of any one of embodiments 1-156, wherein the cosmetic use comprises skin lightening.

158. The topical composition of any one of embodiments 1-157, wherein the cosmetic use comprises skin brightening.

159. The topical composition of any one of embodiments 1-158, wherein the cosmetic use is selected from reducing the number and/or severity of wrinkles, reducing the number and/or severity of fine lines, reducing skin laxity, reducing the number, area, and/or darkness of age spots, reducing the number, area, and/or darkness of stretch marks, reducing the number, area, and/or darkness of spider veins, reducing skin dryness, reducing skin oil buildup, repairing sun damage, alleviating peeling skin, alleviating scaling skin, improving skin tone, reducing skin dullness, reducing appearance of mole, reducing appearance of scar, reducing appearance of birthmark, reducing skin blushing or skin flushing, reducing under-eye dark circle, reducing under-eye bag, skin lightening, skin brightening, or combinations thereof.

160. The topical composition of any one of embodiments 1-159, wherein the cosmetic use is non-medical use.

161. The topical composition of any one of embodiments 1-160, wherein the cosmetic use does not comprise or involve treatment of skin diseases or skin disorders.

### NON-LIMITING EXAMPLES

The following specific, non-limiting examples are to be construed as merely illustrative, and do not limit the present disclosure of the scope of the disclosure. Without further elaboration, it is believed that one skilled in the art can, based on the description herein, utilize the present disclosure to its fullest extent.

### Example 1: Topical DIM Formulation-Emulsion Gel

In this non-limiting example, the topical DIM composition is formulated as an emulsion gel, in which the oil phase contains the DIM compounds and the aquesous phase comprises a hydrogel. Below is a list of the ingredients for the emulsion gel formulation.
Water—aquesous carrier
Glycerin—carrier
DIM—active agent (0.1-10 wt %)
Dicaprylyl carbonate ester—emollient
Ammonium acryloyldimethyltaurate/VP copolymer—rheology modifier
Carbomer—rheology modifier
Polyglyceryl-10 laurate—emulsifier
Levulinic acid—skin conditioner and preservative
Sodium anisate—preservative
Sodium levulinate—skin conditioner and preservative
Sodium hydroxide—pH adjusting agent
Tocopherol—moisturizer/antioxidant

To make the formulation, various ingredients were mixed together until a uniform emulsion gel is obtained.

### Example 2: Topical DIM Formulation-Cream

In this non-limiting example, the topical DIM composition is formulated as an emulsion cream, in which the oil phase contains the DIM compounds and the aquesous phase comprises a base cream. Below is a list of the ingredients for the emulsion cream formulation.
Water—aquesous carrier
Prunus amygdalus dulcis (sweet almond) oil
Dicaprylyl carbonate ester—emollient
Glycerin—carrier
Stearyl alcohol fatty alcohol
Polyglyceryl-3 stearate—emulsifier
Potassium cetyl phosphate—emulsifier
Glyceryl stearate citrate—emulsifier
Xanthan gum—rheology modifier/gum
Levulinic acid—skin conditioner and preservative
Ammonium acryloyldimethyltaurate/VP copolymer rheology modifier—rheology modifier
Sodium anisate—preservative
Sodium levulinate—skin conditioner and preservative
Helianthus annuus (sunflower) seed oil
Sodium hydroxide—pH adjusting agent
Rosmarinus officinalis (rosemary) leaf extract
Tocopherol—moisturizer/antioxidant

To make the formulation, various ingredients were mixed together until a uniform emulsion cream is obtained.

### Example 3: Dermal Bioavailability of DIM

Skin is the outermost largest organ in the body. It protects the body from physical, chemical, and microbial assaults and also prevents water loss from the body. The skin comprises three layers, i.e. epidermis, dermis, and subcutaneous tissue. Epidermis is made up of five layers composed of stratum corneum, stratum lucidum, stratum granulosum, stratum spinosum, and stratum basale. The stratum corneum is the outermost layer and comprise dead keratinocytes, and lamellar granules. The corneocytes and lipids form tight junctions in the stratum corneum like brick and mortar. Stratum corneum acts as a permeation and diffusional barrier for topically applied dosage form and transdermal cosmetic agent (e.g. DIM) delivery. Topical cosmetic agent delivery systems are mostly preferred to deliver the cosmetic agent (e.g. DIM) to the target site with minimal systemic adverse effects.

The bioavailability of the topical and transdermal administered cosmetic agents (e.g. DIM) is affected by various factors. They include permeation through the skin (rate-limiting membrane stratum corneum), physicochemical properties of the cosmetic agent (pKa, logP, solubility, molecular weight), excipients used in the formulation, type of formulation, presence of enzymes in skin structures, application site, and type of the skin. After the topical or transdermal application of the formulation onto the skin, the cosmetic agent must permeate deeper layers by crossing various layers. Various permeability enhancement techniques are employed to increase cosmetic agent transport into the stratum corneum. Consequently, skin cannot be deemed as a single compartment. The estimation of cosmetic agent retained in skin layers is critical in topical and transdermal cosmetic agent delivery. Therefore, there is a need to separate the skin into layers to estimate the cosmetic agent permeation in different layers. Skin bioavailability studies can be very useful for evaluation of topical product's performance and assessment in cosmetic agent permeation-retention in the skin layers.

Many techniques may be used to assess skin bioavailability. Parameters such as Cmax, Tmax, and AUC are extensively assessed in skin bioavailability studies. Assessment of skin bioavailability administered topical formulations is desirable in appraising the products' safety and efficacy. Several techniques have been explored for the estimation of the cosmetic agent in the different skin layers. Using two or three techniques simultaneously provides superior benefits. Skin bioavailability evaluation can be utilized for quality control tool for complex formulations and can provide the road map for newer product development.

### Skin Bioavailability Evaluation Techniques

The skin bioavailability evaluation method is employed to estimate cosmetic agent (e.g. DIM) concentrations in the skin layers. Although the stratum corneum might not be the site of action, it is the rate-limiting membrane for cosmetic agent permeation. The skin bioavailability evaluate is akin to blood and urine pharmacokinetic studies. The skin kinetics depends on the cosmetic agent absorption and the termination of administration. Skin bioavailability evaluation methods assume three mechanisms: i) stratum corneum behavior as the rate-limiting membrane in cosmetic agent absorption; ii) diffusion of the cosmetic agent into the dermis is directly proportional to the concentration of cosmetic agent at the stratum corneum; iii) The cosmetic agent concentrations in the skin layers (stratum corneum, epidermis, dermis) depict the dermatological efficacy of the cosmetic agent. The parameters in skin bioavailability include Cmax (maximum cosmetic agent concentration in the skin layers), Tmax (time required to reach Cmax), and Area under the curve (AUC) in stratum corneum, epidermis, dermis. The Cmax, Tmax and AUC parameters are used to evaluate cosmetic agent's bioavailability in the skin layers. The skin bioavailability profile of the topically applied cosmetic agents is estimated using in-vitro and in-vivo techniques as discussed in the following section.

### Tape Stripping

Tape stripping is the most widely employed technique for the estimation of the cosmetic agent in the stratum corneum. The utilization of tape stripping for quantification of cosmetic agents in the stratum corneum was proposed in the draft guidance by the United States Department of Health and Human Services in 1998 to assess the bioequivalence of the topical products. This method can be utilized in both in-vitro and in-vivo cosmetic agent permeation studies for topical delivery systems applied to the skin. It is uncomplicated, easily implemented for quantification of cosmetic agent retained in the stratum corneum. This technique is minimally invasive in the in-vivo conditions (Preclinical and clinical studies). It involves removing the stratum corneum layer one after another using adhesive tape strips. The tape strip is applied with the pressure using a roller to ensure uniform and proper adhesion onto the skin surface. The tape strip's application pressure should maintain uniform throughout the tape stripping process to get reproducible results. The collected tape strips are added to the solvent of interest in which the cosmetic agent is highly soluble to extract the cosmetic agent from the adhesive. The cosmetic agent extracted is subjected to centrifugation and quantified using a validated analytical technique. The typical limitations with the tape stripping technique is the interference of adhesive, and the number of strips required to remove the stratum corneum is varied from person to person and site of application. There is a requirement of validating the procedure before the utilization of the process in preclinical and clinical studies. The number of stratum corneum layers differs in animals (mice, rat, rabbit, guinea pig, goat, pig) and humans.

The tape stripping technique is validated using the weighing method or the protein estimation method. In weighing method, the tape strip's weight is noted before and after tape stripping, and the protein levels of corneocytes are measured to determine the stratum corneum layers in the skin. Olesen et al. reported that protein removal was decreased with an increase in tape stripping depth. The study results revealed that tape stripping can be used to skin barrier research without discomfort and scars compared to skin biopsies.

### Microdialysis and Open-Flow Microperfusion

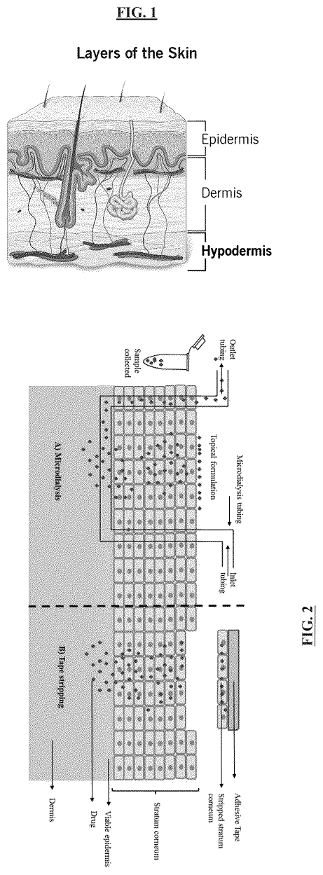

A microdialysis and open-flow microperfusion techniques are employed to assess the bioequivalence of the topical cosmetic product. The cosmetic agent concentration is determined in the dermis and hypodermis by implanting a semipermeable membrane probe guided by a needle. The probe is perfused with the sterile buffer, which mimics the blood vessels, equilibrates by interstitial fluids, and the cosmetic agent diffuse from tissues to the buffer. Isotonic saline/Ringer's lactate solution is perfused at the rate of 1-5 μL. FIG. 2 depicts the microdialysis and tape striping technique.

Figure below illustrates microdialysis and tape striping technique: (a) In microdialysis technique, the dialysis tubing is inserted into tissues and physiological solution (saline/ringer solution) is perfused at slow speed. The permeated cosmetic agent diffuse into the dialysis tube are collected for estimation of cosmetic agent; (B) in tape stripping technique, the adhesive tape is used to strip the stratum corneum. The adhesive tape strip with stratum corneum is added to the solvent with high cosmetic agent solubility. The cosmetic agent is extracted, and the cosmetic agent is estimated.

### Vasoconstrictor Assay

Vasoconstrictor assay is also termed as the skin blanching assay, which is only limited to the corticosteroid formulations' bioavailability assessment. After applying corticosteroids, the local vasoconstriction can be measured by visual examination, chromametry, and analysis using digital imaging. In chromametry, the white light gets reflected into three different constraints red-green, yellow-blue, and light- dark. Using a digital image of 0.5 cm2 with 300 dots per inch is analyzed for the above constraints. Visual inspection is an alternative to chromametry; the visual responses are given in the range of 0-4. Whereas 0 indicates no blanching, and required further no blanching, whereas numbers from 1 to 4 show the increasing array of blanching (absent=0, faint=1, faint-moderate=2, moderate-strong=3, and strong-intense blanching=4) practical application.

### Microscopic and Spectroscopic Methods

Along with the above methods, microscopic and spectroscopic techniques have been widely explored for the assessment of cosmetic agent distribution. In microscopy analysis techniques, the dye (fluorescein, Nile red, and 5-bromodeoxyuridine, coumarin c6) is utilized for the assessment of the distribution and penetration into deeper layers of the skin. The dye may not exhibit similar behavior as a cosmetic agent, which is the major limitation of the technique. In spectroscopic techniques, Attenuated Total Reflectance (ATR) or Fourier-Transform Infrared Spectroscopy (FTIR) is employed to estimate the modifications in the stratum corneum modifications. The conformation of proteins in outer skin layers, an assortment of lipids, treatment with chemical enhancers, and employing physical methods may modify permeability. The shift in the infrared bands illustrates the relative alterations between treated and untreated skin. The combination of Raman spectroscopy and confocal microscopy attracted great attention in estimating cosmetic agent distribution in skin samples. The interference of water in Raman spectroscopy is null; it works on the principle of inelastic scattering of monochromatic light from a laser beam. The chemical structure, electronic configuration, and molecular bonds of detailed compound information are obtained using Raman spectroscopy.

### Example 4: Topical Application Increases Maximal Plasma Concentration (Cmax) and Dermal Bioavailability (AUC) of DIM in the Skin

The objective of this study is to determine the bioavailability of a diindolylmethane (DIM) formulation in skin following single and multiple doses of DIM, administered either by topical route or by oral route. The DIM topical formulation for this study is an exemplary formulation prepared according to Example 1 and/or Example 2. The DIM oral formulation for this study is prepared according to U.S. Pat. No. 9,180,117.

Participants are grouped into (a) Group 1: DIM administered by oral route and (b) Group 2: DIM administered by topical route. Participants are administered, orally or topically, respectively, a single 10 mg dose of the DIM formulation. These participants are not exposed to DIM earlier and thus are naïve subjects. Mean dermal Cmax for the topical administration is assessed and compared to mean dermal Cmax for the oral administration.

The dermal bioavailability of DIM is derived from AUC after the single 10 mg dose, topically or orally, in the naïve subjects. Mean dermal AUC for the topical administration is assessed and compared to mean dermal AUC for the oral administration.

### Example 5: Clinical Study on Cosmetic Benefits of Using Topical DIM

The objective of this study was to assess the cosmetic effectiveness and tolerability of topical DIM.

### METHOD

### Participants, Procedure and Product

Twenty-six men and women, aged 18 to 68 years (average 36 yrs), were recruited for this trial. Participants were healthy, from a range of ethnic groups, and had a range of skin conditions: 5 participants had moderate facial acne vulgaris, 13 had moderate atopic dermatitis, 3 had psoriasis and 5 had other skin conditions including hyperpigmentation.

Participants were excluded if they had used oral retinoids in the 3 months prior to screening, topical retinoids in the one month prior to screening, or were pregnant or lactating. If on hormonal contraception the prescription needed to remain the same over the 3 months prior to screening and for the duration of the trial. The use of nicotine was prohibited in the month before screening, and the duration of the trial.

For 2 weeks prior to the onset of testing and for the duration of the trial, participants were instructed not to use prescribed medication, topical medication and/or self-tanning products, have any facial procedures/facials/chemical peels/dermabrasion, avoid excessive UV light exposure, take food supplements except for medically prescribed supplements such as iron or vitamin B12, or drink more than 14 units of alcohol per week. They were also required to not make any changes to their skincare routine. The day before testing they were allowed to follow their normal skincare routine, however on the day of testing, they were instructed to only wash their face/neck or affected areas with water and not wear any cosmetics.

### Assessment

This study was conducted over 16 weeks, with assessments at baseline (week 0) and weeks 4, 8 and 16. Assessment consisted of physiological measurements, imaging and questionnaires. Control sites were measured in all participants.

### Physiological Measurements

The Courage & Khazaka™ equipment was used to measure properties of the skin. Courage & Khazaka™ are worldwide leaders in the production of dermatology testing devices.
Direct measurement of skin using Courage & Khazaka™ probes:
   Hydration in the stratum corneum is assessed with a Corneometer® which measures the capacitance of the di-electric properties of the stratum corneum. Hydration is measured in arbitrary units (AU) between 0 and 120, with very dry being <30, dry being 30-40 and normal >40.
   Transepidermal water loss (TEWL) is assessed with a Tewameter® which uses open-chamber evaporimeters to measure TEWL. This together with the electrical method used by the Corneometer are the preferred techniques for measuring the water balance in the stratum corneum. TEWL is measured in g/h/m², with values >20 defined as increased.
   Melanin and erythema (haemoglobin) are assessed with a Mexameter®, which emits 3specific wavelengths of light and measures the reflectance of skin (light reflected back). Erythema is grouped into: none <170, minimal 170-330, diffuse redness 330-450, high 450-750 and extreme >570. Melanin varies per skin type. Both erythema and melanin are measured with arbitrary units (AU), between 0-999.
   Sebum levels are assessed with the Sebumeter®, which uses grease spot photometry. Sebum is measured in μg/cm². On the forehead and t-zone, a value of <100 is defined as dry/less sebum, 100-180 as normal, and >180 as oily.

### Imaging

High-resolution imaging of the skin surface with the Courage and Khazaka™ Visioscan®, a unique high resolution, UVA light video camera. This allows assessment of wrinkle depth, skin smoothness and skin roughness.
Imaging of the full face using the Canfield® Visia equipment. This uses IntelliFlash®, cross-polarized and UV lighting to record and measure surface and subsurface skin conditions.

### Questionnaire

A self-assessment questionnaire was administered after initial application of the product, and at the end of weeks 1, 2, 4, 8, 12 and 16.

### Statistical Analysis

### Subject Characteristics

Differences in skin parameters, including stratum corneum hydration, elasticity, erythema, melanin, TEWL and sebum production were analysed using one-way ANOVA. This analysis evaluated the effects of treatment group and time (Weeks 0, 4, 8, and 16). Additionally, pooled two-sampled t-tests were conducted to compare differences between groups at each time point.

### Normality Testing

The normality of the data sets was evaluated using the Data Analysis Toolpak in Microsoft Excel to guide the selection of appropriate statistical methods. Levene's test showed that all data sets followed a normal distribution, enabling the use of one-way ANOVA for comparing independent groups.

ANOVA and t-Tests

Comparisons between treatment and control areas within each group were made using one-way ANOVA. Additionally, t-tests were applied to assess differences across time points (Weeks 0, 4, 8, and 16) within each group.

The effects of Cream versus Emulgel formulations on skin parameters were also compared. One-way ANOVA was used to analyse differences between the Cream and Emulgel formulations at various time points, followed by independent t-tests to assess changes between specific time periods.

Additionally, comparisons between DIM concentrations (1% vs. 2.5%) were conducted to assess whether different concentrations had an effect on skin parameters in each group. One-way ANOVA was used to evaluate differences between the 1% and 2.5% formulations within each group across the time points, with independent t-tests performed to compare differences between each time period.

### RESULTS

A total of 26 participants were included in the trial: 17 female and 9 male, aged 18 to 68 years (average 36 years). All participants completed 8 weeks, and 8 completed the full 16 weeks.

TABLE 1
Measurement of various skin parameters of treated and control
skin at weeks 0 (baseline), 4, 8 and 16 for all participants.

| | | Week 0 | Week 4 | Week 8 | Week 16 |
|---|---|---|---|---|---|
| TEWL (g/h/m²) | Treated | 28.07 | 22.47* | 24.97 | 20.41** |
| | Control | 16.29 | 16.40 | 18.22 | 20.33 |
| Hydration (AU) | Treated | 25.08 | 31.93 | 33.57*** | 33.35 |
| | Control | 34.77 | 33.79 | 34.26 | 37.65 |
| Erythema (AU) | Treated | 383.88 | 356.92 | 366.53 | 319.34**** |
| | Control | 255.30 | 245.05 | 240.08 | 219.61 |
| Melanin (AU) | Treated | 326.06 | 340.10 | 336.53 | 278.19 |
| | Control | 243.01 | 220.53 | 223.52 | 163.17 |
| Sebum (μg/cm²) | Treated | 40.91 | 20.82 | 21.94 | 17.43 |
| | Control | 16.62 | 21.00 | 6.54 | 39.20 |

TEWL = trans-epidermal water loss;
*week 0 vs week 4, p = 0.039;
**week 0 vs week 16, p = 0.048;
***week 0 vs week 8, p = 0.020;
****week 0 vs week 16, p = 0.003.

### Hydration

### Physiological Measurements

TEWL decreased by 19.9% from week 0 to week 4 (p=0.039) and by 27.3% from week 0 to week 16 (p=0.048). All participants starting with an abnormal TEWL showed a 29.7% reduction by week 4 (p=0.002) and a 40.6% reduction by week 16 (p=0.007).

Hydration increased by 33.9% from week 0 to week 8 (p=0.020) and remained stable to week 16. There were no changes in TEWL or hydration in the control group. Data is shown in table 1.

### Questionnaire Results

88% agree—after 2 weeks of continuous use, the product hydrates skin (n=24)
83% agree—after 4 weeks of continuous use, the product hydrates skin (n=24)
90% agree—after 16 weeks of continuous use, the product hydrates skin (n=10)

### Redness/Erythema

### Physiological Measurements

Erythema decreased by 16.8% from week 0 to week 16 (p=0.003). There were no changes in erythema in the control group. Data is shown in table 1.

### Questionnaire Results

78% agreed—after 8 weeks of continuous use, skin redness decreased (n=23)
100% of participants with acne agreed—after 1 week of continuous use, skin redness decreased (n=4)
100% of participants with acne strongly agreed—after 8 weeks continuous use, skin redness decreased (n=4)
100% of participants with psoriasis strongly agree—after 8 weeks continuous use, skin redness decreased (n=3)
100% of participants with psoriasis strongly agree—after 12 weeks continuous use, skin redness decreased (n=2)

### Imaging Evaluation

FIG. 3 illustrates the effect of topical DIM on reduction of redness/erythema.

### Skin tone

### Questionnaire Results

100% of participants with acne agreed—after 1 week of continuous use, pigmentation decreased (n=3)
100% of participants with acne agreed—after 8 weeks continuous use, acne scarring improved (n=4)
100% of participants with atopic dermatitis strongly agreed—after 16 weeks of continuous use, pigmentation decreased (n=3)
100% of participants with psoriasis strongly agree—after 12 weeks continuous use, pigmentation decreased (n=2)

### Imaging Evaluation

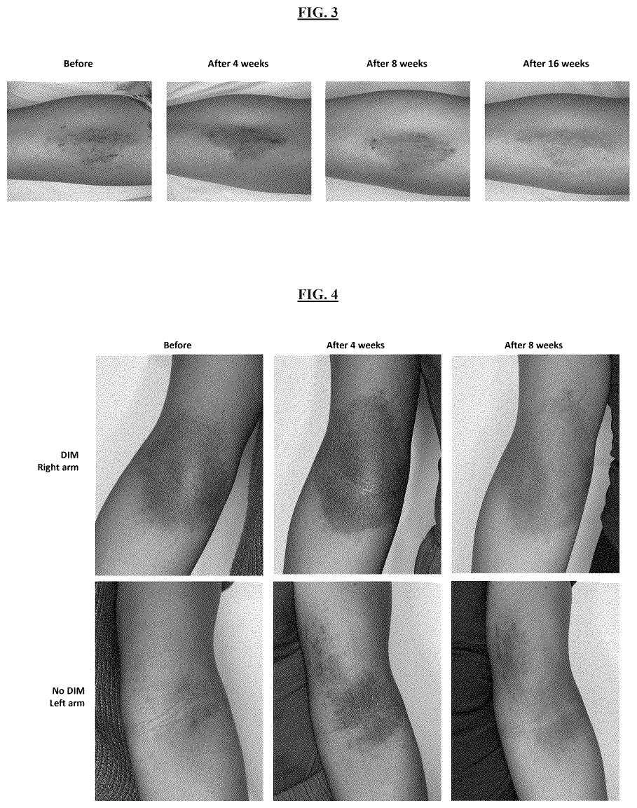

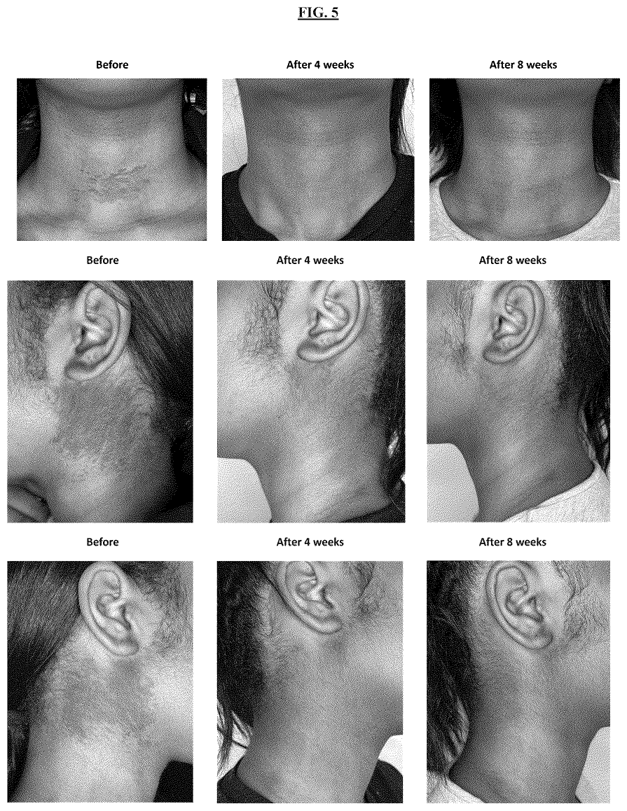

FIG. 4 illustrates a comparison of reduction of pigmentation/skin tone between topical DIM and no DIM groups.

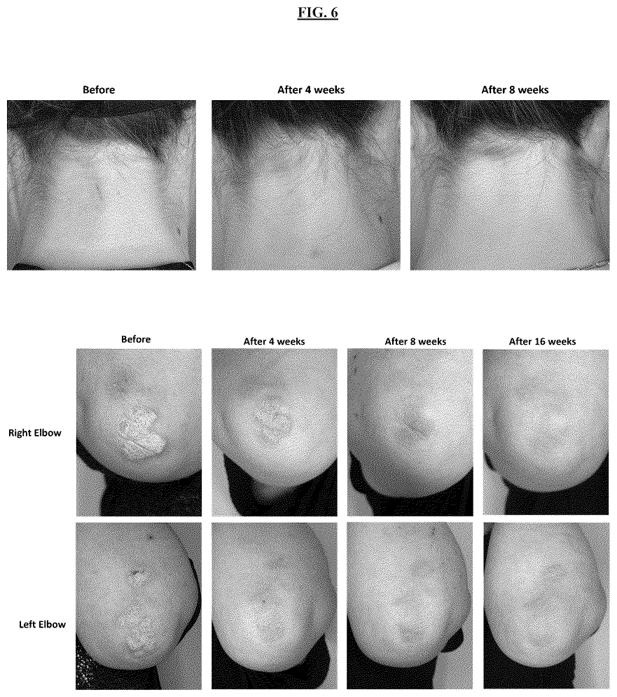

FIG. 5 illustrates additional examples of reduction of pigmentation/skin tone by topical DIM.

### Skin Oiliness

### Physiological Measurements

Sebum decreased in the treated group, while it increased in the control group, however both of these were non-significant. Data is shown in table 1.

### Skin Appearance: Texture and Smoothness

### Questionnaire Results

78% of participants agreed—after 4 weeks continuous use, skin texture improved (n=23)
70% of participants agreed—after 16 weeks continuous use, skin texture improved (n=10)
100% of participants with acne agreed—after 4 weeks continuous use, skin texture improved (n=4)
100% of participants with acne strongly agreed—after 8 weeks continuous use, skin texture improved (n=4)
100% of participants with psoriasis strongly agreed-after 12 weeks continuous use, skin texture improved (n=2)

### Imaging Evaluation

FIG. 6 illustrates examples of improvement of skin texture and smoothness by topical DIM.

### Skin Appearance: Healthy-Looking Skin

### Questionnaire Results

78% agreed—after 1 week of continuous use, my skin looked healthier (n=23)
78% agreed—after 4 weeks of continuous use, my skin looked healthier (n=23)
78% agreed—after 8 weeks of continuous use, my skin looked healthier (n=23)
100% of participants with acne strongly agreed—after 8 weeks continuous use, my skin looked healthier (n=4)
100% of participants with psoriasis strongly agreed—after 1 week continuous use, my skin looked healthier (n=3)
100% of participants with psoriasis strongly agreed—after 2 week continuous use, my skin looked healthier (n=3)
100% of participants with psoriasis strongly agreed—after 12 week continuous use, my skin looked healthier (n=2)

### Skin Soothing and Calming

### Questionnaire Results

88% agree—the product instantly soothes skin (n=24)
88% agree—the product instantly calms skin (n=24)

### Cream vs Emulgel

11 participants used the cream, while 15 used the emulgel.

At baseline, erythema was higher in cream group compared to emulgel (p=0.011). Both cream and emulgel led to decreased erythema, however this was only significant in the cream group (week 16, p=0.006). Both cream and emulgel led to increased hydration, however this was only significant in the emulgel group (week 4, p=0.052; week 8, p=0.017).

Skin measurements shown in table 2.

In summary, both cream and emulgel led to decreased erythema and increased hydration. However, due to the small sample sizes, it is impossible to make a conclusive comparison.

TABLE 2
Measurement of various skin parameters treated by cream and Emulgel
at weeks 0 (baseline), 4, 8 and 16 in combined skin conditions

| | | Week 0 | Week 4 | Week 8 | Week 16 |
|---|---|---|---|---|---|
| TEWL (g/h/m²) | Cream | 25.48 | 20.69 | 22.82 | 19.25 |
| | Emulgel | 30.57 | 24.19 | 27.04 | 22.72 |
| Hydration (AU) | Cream | 29.54 | 35.59 | 36.43 | 35.35 |
| | Emulgel | 20.78 | 28.40* | 30.81** | 24.60 |
| Erythema (AU) | Cream | 410.87 | 379.18 | 388.90 | 329.60*** |
| | Emulgel | 357.89 | 335.48 | 344.99 | 300.29 |
| Melanin (AU) | Cream | 386.71 | 402.69 | 403.16 | 276.49 |
| | Emulgel | 267.65 | 279.83 | 272.38 | 281.36 |
| Sebum (μg/cm²) | Cream | 37.13 | 14.73 | 20.40 | 17.57 |
| | Emulgel | 46.22 | 26.17 | 24.39 | 19.83 |

TEWL = trans-epidermal water loss;
*week 0 vs week 4, p = 0.052;
**week 0 vs week 8, p = 0.017;
***week 0 vs week 16, p = 0.006.

### 1% vs 2.5% DIM Concentrations

9 participants used the 1% product, while 17 used the 2.5% product.

The 1% product led to a decrease in TEWL (week 16, p=0.048), and an increase in hydration (week 16, p=0.024), while the 2.5% product led to a non-significant increase in hydration. Skin measurements are shown in table 3.

TABLE 3
Measurement of various skin parameters treated by
1% and 2.5% DIM concentrations at weeks 0 (baseline),
4, 8 and 16 in combined skin conditions

| | | Week 0 | Week 4 | Week 8 | Week 16 |
|---|---|---|---|---|---|
| TEWL (g/h/m²) | 1% | 22.63 | 16.98 | 18.22 | 14.83* |
| | 2.5% | 30.48 | 24.99 | 28.07 | 24.59 |
| Hydration (AU) | 1% | 25.99 | 38.44 | 37.32 | 45.71** |
| | 2.5% | 24.14 | 29.51 | 32.25 | 23.24 |
| Erythema (AU) | 1% | 397.73 | 352.62 | 356.13 | 321.13 |
| | 2.5% | 379.39 | 361.97 | 373.54 | 317.88 |
| Melanin (AU) | 1% | 459.92 | 446.16 | 440.75 | 292.19 |
| | 2.5% | 277.80 | 296.82 | 292.15 | 266.74 |
| Sebum (μg/cm²) | 1% | 32.20 | 14.20 | 22.70 | 21.20 |
| | 2.5% | 46.39 | 23.91 | 22.52 | 17.00 |

TEWL = trans-epidermal water loss;
*week 0 vs week 16, p = 0.048;
**week 0 vs week 16, p = 0.024.

In summary, both 1% and 2.5% products led to improvements in TEWL and hydration. However, due to the small sample sizes, it is impossible to make a conclusive comparison.

### Safety

2 participants experienced a flare up of atopic dermatitis. Both patients were using the emulgel. One participant experienced a flare up of atopic dermatitis as the product was not hydrating enough and so had an increase in itch which led to scratching and excoriation. She stopped using the test product for a few days, then restarted adding an emollient to her daily regimen to improve hydration and her atopic dermatitis settled, with no further flare ups with continued use.

The second participant experienced an increase in itch, pain and redness in her legs 2 weeks after starting the trial. However, this coincided with a time of year when her skin typically flares. She continued using the product and the skin on her legs settled. She reported another reaction 9 weeks into the trial, where her face became puffy and red after she inadvertently touched it with product still on her hands. This worsened over several days and then slowly settled. She only reported this to us a couple weeks later, so the detailed history is unknown, and it is unclear as to whether this was a reaction to the topical product or not. She continued to use the product on her legs throughout with no further reactions.

Overall, both emugel and cream products demonstrated good safety profile after topical applications.

### Conclusion

The use of topical DIM led to several cosmetic benefits including improved skin hydration, decreased appearance of redness, and evened skin tone. It had a soothing and calming effect, improved skin texture, smoothness and softness, and promoted healthier looking skin. It was also safe to use.

Both cream and emulgel led to decreased erythema and increased hydration, and both 1% and 2.5% products led to improvements in TEWL and hydration. Even though this is an preliminary study with a small sample size, the results are extremely promising.

What is claimed is:

1. A topical composition for cosmetic use in a subject in need thereof, wherein the composition comprises from about 0.1 wt % to about 10 wt % of emulsified substituted or unsubstituted diindolylmethane and a pharmaceutically acceptable carrier, and wherein the composition does not cause irritation or allergic reaction after topical application.

2. The topical composition of claim 1, wherein the composition comprises from about 0.1 wt % to about 8.0 wt % of substituted or unsubstituted diindolylmethane.

3. The topical composition of claim 1, wherein the composition comprises from about 0.1 wt % to about 5.0 wt % of substituted or unsubstituted diindolylmethane.

4. The topical composition of claim 1, wherein the composition comprises from about 0.3 wt % to about 3.0 wt % of substituted or unsubstituted diindolylmethane.

5. The topical composition of claim 1, wherein the composition comprises from about 0.5 wt % to about 2.5 wt % of substituted or unsubstituted diindolylmethane.

6. The topical composition of claim 1, wherein the composition comprises from about 0.8 wt % to about 1.2 wt % of substituted or unsubstituted diindolylmethane.

7. The topical composition of claim 1, wherein the composition comprises from about 1.0 wt % of substituted or unsubstituted diindolylmethane.

8. The topical composition of claim 1, wherein the composition comprises from about 2.0 wt % to about 3.0 wt % of substituted or unsubstituted diindolylmethane.

9. The topical composition of claim 1, wherein the composition comprises about 2.5 wt % of substituted or unsubstituted diindolylmethane.

10. The topical composition of claim 1, wherein the substituted or unsubstituted diindolylmethane is unsubstituted diindolylmethane.

11. The topical composition of claim 1, wherein the substituted or unsubstituted diindolylmethane is unsubstituted 3,3′-diindolylmethane.

12. The topical composition of claim 1, wherein the composition is essentially free of any retinoid compound.

13. The topical composition of claim 1, wherein the composition is essentially free of any cosmetically active agent other than the substituted or unsubstituted diindolylmethane.

14. The topical composition of claim 1, wherein the cosmetic use is selected from reducing the number and/or severity of wrinkles, reducing the number and/or severity of fine lines, reducing skin laxity, reducing the number, area, and/or darkness of age spots, reducing the number, area, and/or darkness of stretch marks, reducing the number, area, and/or darkness of spider veins, reducing skin dryness, reducing skin oil buildup, repairing sun damage, alleviating peeling skin, alleviating scaling skin, improving skin tone, reducing skin dullness, reducing appearance of mole, reducing appearance of scar, reducing appearance of birthmark, reducing skin blushing or skin flushing, reducing under-eye dark circle, reducing under-eye bag, skin lightening, skin brightening, or combinations thereof.

15. The topical composition of claim 1, wherein the cosmetic use comprises enhancing skin appearance.

16. The topical composition of claim 1, wherein the cosmetic use comprises reducing skin dryness.

17. The topical composition of claim 1, wherein the cosmetic use comprises reducing skin oil buildup.

18. The topical composition of claim 1, wherein the cosmetic use comprises improving skin tone.

19. The topical composition of claim 1, wherein the cosmetic use comprises reducing skin blushing or skin flushing.

20. The topical composition of claim 1, wherein the cosmetic use is non-medical use, and optionally, wherein the cosmetic use does not comprise or involve treatment of skin diseases or skin disorders.
